(19) United States
(12) Patent Application Publication
(10) Pub. No.: US 2025/0316340 A1
(43) Pub. Date: Oct. 9, 2025

(54) MONITORING ASSAY PERFORMANCE

(71) Applicant: Genentech, Inc., South San Francisco, CA (US)

(72) Inventor: Maxime Steve USDIN, San Diego, CA (US)

(21) Appl. No.: 19/242,851

(22) Filed: Jun. 18, 2025

Related U.S. Application Data
(63) Continuation of application No. PCT/US2023/084655, filed on Dec. 18, 2023.
(60) Provisional application No. 63/433,598, filed on Dec. 19, 2022.

(51) Int. Cl. G16B 40/30; G06F 17/18

(52) U.S. Cl. CPC G16B 40/30; G06F 17/18

(57) ABSTRACT

A plurality of instances of an assay may be performed to obtain a plurality of concentration data points. Next, a significant change point that corresponds to a location in the plurality of concentration data points at which one or more statistical characteristics of the plurality of concentration data points change by more than a threshold value may be identified. The significant change point may be correlated with one or more assay parameters associated with the assay by identifying an instance of performing the assay that corresponds to the location of the significant change point in the plurality of concentration data points. Based on the correlation, the cause for the change in the one or more statistical characteristics of the plurality of concentration data points at the identified significant change point may be determined.

### CROSS-REFERENCE TO RELATED APPLICATIONS

This application is a continuation of International Patent Application No. PCT/US2023/084655, filed on Dec. 18, 2023, which claims priority to U.S. Provisional Patent Application No. 63/433,598, entitled “Monitoring Assay Performance,” filed on Dec. 19, 2022, the disclosure to which is incorporated herein by reference in its entirety.

### FIELD

The present disclosure relates generally to techniques for monitoring and evaluating the performance of analytic biological procedures. More specifically, the present disclosure relates to methods for identifying and evaluating the significance of changes in data collected during an analytic biological procedure.

### BACKGROUND

Taking repeated measurements of a process, or one or more aspects thereof, over time has the potential to provide valuable insights into changes occurring within the process. However, techniques for the analysis of such data to confidentially identify process change is currently lacking.

Sources of process data for analyzing process changes are numerous. For example, in the production of a biologic therapeutic, such as an antibody, immunoassays are useful for tracking the concentration of the biologic between different production runs. The production of a biologic therapeutic is very complex and involves numerous reagents and components (such as living cells), instruments, manufacturing and testing environments, and human operators, all of which are susceptible to change over time. Careful monitoring of biologic therapeutic manufacturing processes, including monitoring of assays using controls, is essential to the production quality control of such therapeutics. An additional example can be found in the analysis of digital biomarkers, such as from measurements of the human body performed over time from medical and/or consumer smart devices, e.g., a smart watch. The analysis of these biomarkers produce complex multi-parameter data, including linked information regarding heart rate, glucose level, blood-oxygen content, GPS coordinates, gyroscope data, and environmental conditions.

Current techniques for evaluating such data, which is often irregularly spaced, noisy, multi-dimensional, and contains limited ground truths, involve tedious manual processes with a lack of an established metric for identifying process outliers. Moreover, current techniques only enable the identification of process deviations well after the process is performed, thus making the rectification of an issue undesirably slow.

### SUMMARY

As described above, process data often includes a time series of repeated measurements of a process. Provided are methods for determining the causes of statistical changes in process data by identifying locations in process data where significant changes in the data have occurred. The methods may allow laboratory analysts to efficiently identify periods of time when important shifts in the data have occurred. Once the relevant periods of time have been identified, the analysts can investigate potential causes of the shifts in the data by evaluating parameters associated with the measurements taken during the identified periods of time.

In some embodiments, the methods described can both identify locations in process data where changes in the data have occurred and validate the significance of said changes with respect to the data set as a whole. Once the potential locations are pinpointed algorithmically, the significance of each potential location may be validated by quantifying statistical changes in the data surrounding the location. This quantification of the statistical changes can be used determine whether the changes that occur at that location are significant—and, therefore, potentially indicative of measurement error or other causes worth evaluating—or insignificant (e.g., the result of random statistical fluctuations). By validating the significance of the identified changes in the process data, the methods provided herein can, in some embodiments, help analysts spend time investigating important changes that have a high probability of impacting the results of the process being measured.

An example of a method for determining a cause of a significant statistical change in a plurality of concentration data points obtained from an assay may comprise performing a plurality of instances of the assay to obtain the plurality of concentration data points, receiving assay information comprising the plurality of concentration data points and a plurality of assay parameters, wherein each assay parameter of the plurality of assay parameters is associated with an instance of the plurality of instances of performing the assay, identifying a significant change point that corresponds to a location in the plurality of concentration data points at which one or more statistical characteristics of the plurality of concentration data points change by more than a threshold value, correlating one or more assay parameters of the plurality of assay parameters with the identified significant change point by identifying an instance of the plurality of instances of performing the assay that corresponds to the location of the significant change point in the plurality of concentration data points, and determining the cause for the change in the one or more statistical characteristics of the plurality of concentration data points at the identified significant change point based on the correlation between the one or more assay parameters and the significant change point.

In some embodiments of the method, the assay is configured to measure a concentration of an analyte in a sample.

In some embodiments of the method, the analyte is a therapeutic analyte.

In some embodiments of the method, the analyte is a therapeutic polypeptide.

In some embodiments of the method, the analyte is an antibody or a fragment thereof.

In some embodiments of the method, the sample is a cell culture sample or a derivative thereof.

In some embodiments of the method, the assay is an immunoassay.

In some embodiments of the method, the assay is a competitive assay.

In some embodiments of the method, the assay is a non-competitive assay.

In some embodiments of the method, the assay is a non-homogenous assay.

In some embodiments of the method, the assay is a homogenous assay.

In some embodiments of the method, the assay is an ELISA assay.

In some embodiments of the method, the ELISA assay is a direct ELISA assay.

In some embodiments of the method, the ELISA assay is a sandwich ELISA assay.

In some embodiments of the method, the ELISA assay is a competitive ELISA

In some embodiments of the method, performing the plurality of instances of the assay comprises performing two or more of the plurality of instances at two or more times.

In some embodiments of the method, the two or more times constitute a time course of at least about one week.

In some embodiments of the method, performing the plurality of instances of the assay comprises performing two or more of the plurality of instances simultaneously.

In some embodiments of the method, the plurality of concentration data points comprises data points pertaining to a concentration a target analyte.

In some embodiments of the method, the plurality of concentration data points comprises data points pertaining to a concentration of a control.

In some embodiments of the method, the control is a negative control, non-specific binding control, blank control, detection antibody control, negative matrix control, or positive control.

In some embodiments of the method, the plurality of concentration data points comprises data points pertaining to a solution concentration.

In some embodiments of the method, the plurality of concentration data points comprises data points pertaining to an absolute amount of a target analyte.

In some embodiments of the method, the plurality of concentration data points comprises data points pertaining to a measurement associated with concentration.

In some embodiments of the method, the measurement associated with concentration is an optical density (OD) measurement.

In some embodiments of the method, the plurality of concentration data points comprises data points pertaining to a mean, lowest standard deviation mean, highest standard deviation mean, or middle control concentration.

In some embodiments of the method, the significant change point reflects inter-assay variability.

In some embodiments of the method, the significant change point reflects intra-assay variability.

In some embodiments of the method, two or more concentration data points of the plurality of concentration data points are in the same format.

In some embodiments of the method, the one or more assay parameters correlated with the significant change point comprise an environmental factor.

In some embodiments of the method, the environmental factor comprises a temperature, humidity, light, or contaminant.

In some embodiments of the method, the one or more assay parameters correlated with significant change points comprise an equipment factor.

In some embodiments of the method, the equipment factor comprises a reagent change, reagent aging, reagent expiration, reagent contamination, reagent failure, hardware change, hardware aging, hardware contamination, hardware failure, instrument change, instrument failure, or instrument calibration.

In some embodiments of the method, the one or more assay parameters correlated with the significant change point comprise a human factor associated with one or more humans who performed or assisted in performing the assay.

In some embodiments of the method, the human factor comprises performance variability, performance error, or operator change.

In some embodiments of the method, identifying the significant change point comprises determining an expected change point population in the plurality of concentration data points.

In some embodiments of the method, identifying the significant change point comprises selecting a first segment of concentration data points of the plurality of concentration data points, determining a first median value associated with the first segment of concentration data points, selecting a second segment of concentration data points of the plurality of concentration data points, wherein the concentration data points in the second segment are consecutive to the concentration data points in the first segment, determining a second median value associated with the second segment of concentration data points, comparing the first median value to the second median value, and determining, based on the comparison between the first median value and the second median value, whether a candidate change point is located between the first segment and the second segment.

In some embodiments of the method, the first segment and the second segment comprise at least a threshold number of concentration data points.

In some embodiments, the method comprises receiving the threshold number of concentration data points from a user.

In some embodiments of the method, the threshold number of concentration data points is determined based on the assay.

In some embodiments, the method comprises, for the first segment and the second segment, generating one or more mean values, generating one or more data point clusters, wherein each data point cluster is associated with a mean value of the one or more mean values and comprises concentration data points in the segment that are closest to the associated mean value, updating the mean value for each data point cluster of the one or more data point clusters, wherein updating the mean value for a data point cluster comprises identifying a centroid of the data point cluster, and iteratively repeating the steps of generating one or more data point clusters and updating the mean values for each data point cluster until the mean values for each data point cluster no longer change.

In some embodiments, the method comprises generating one or more data point clusters within each of the first segment and the second segment, wherein the concentration data points in each data point cluster are normally distributed and have a unique mean value and a unique standard deviation value.

In some embodiments, the method comprises identifying, for the first segment and the second segment, a principal data point cluster of the one or more data point clusters, wherein the principal data point cluster comprises at least a threshold percentage of a total number of concentration data points in the segment.

In some embodiments, the method comprises determining, for the candidate change point, a divergence value, wherein the divergence value measures a statistical difference between the principal data point cluster in the first segment and the principal data point cluster in the second segment.

In some embodiments of the method, the divergence value is a Jensen-Shannon divergence.

In some embodiments, the method comprises determining, for the candidate change point, a median change value, wherein the median change value measures a difference between the first median value associated with the first segment and the second median value associated with the second segment.

In some embodiments, the method comprises determining whether the one or more statistical characteristics of the plurality of concentration data points change by more than a threshold value by determining a weighted combination of the divergence value and the median change value.

In some embodiments of the method, the weighted combination of the divergence value and the median change value is characterized by a weight parameter.

In some embodiments, the method comprises receiving the weight parameter from a user.

In some embodiments of the method, the weight parameter is determined based on the assay.

In some embodiments, the method comprises performing a second plurality of instances of the assay after determining the cause for the change in the one or more statistical characteristics of the plurality of concentration data points at the identified significant change point.

In some embodiments of the method, the second plurality of instances of the assay are performed with assay parameters that match the one or more assay parameters that are correlated with the identified significant change point.

In some embodiments of the method, the second plurality of instances of the assay are performed with assay parameters that match assay parameters associated with instances of performing the assay that occurred prior to the identified significant change point.

In some embodiments, the method comprises deleting one or more concentration data points of the plurality of concentration data points that correspond to instances of the plurality of instances of performing the assay that occurred after the identified significant change point.

An example of a system for determining a cause of a significant statistical change in a plurality of concentration data points obtained from an assay may comprise one or more processors configured to receive assay information comprising the plurality of concentration data points obtained by performing a plurality of instances of the assay and a plurality of assay parameters, wherein each assay parameter of the plurality of assay parameters is associated with an instance of the plurality of instances of performing the assay, identify a significant change point that corresponds to a location in the plurality of concentration data points at which one or more statistical characteristics of the plurality of concentration data points change by more than a threshold value, correlate one or more assay parameters of the plurality of assay parameters with the identified significant change point by identifying an instance of the plurality of instances of performing the assay that corresponds to the location of the significant change point in the plurality of concentration data points, and determine the cause for the change in the one or more statistical characteristics of the plurality of concentration data points at the identified significant change point based on the correlation between the one or more assay parameters and the significant change point.

In some embodiments of the system, the assay is configured to measure a concentration of an analyte in a sample.

In some embodiments of the system, the analyte is a therapeutic analyte.

In some embodiments of the system, the analyte is a therapeutic polypeptide.

In some embodiments of the system, the analyte is an antibody or a fragment thereof.

In some embodiments of the system, the sample is a cell culture sample or a derivative thereof.

In some embodiments of the system, the assay is an immunoassay.

In some embodiments of the system, the assay is a competitive assay.

In some embodiments of the system, the assay is a non-competitive assay.

In some embodiments of the system, the assay is a non-homogenous assay.

In some embodiments of the system, the assay is a homogenous assay.

In some embodiments of the system, the assay is an ELISA assay.

In some embodiments of the system, the ELISA assay is a direct ELISA assay.

In some embodiments of the system, the ELISA assay is a sandwich ELISA assay.

In some embodiments of the system, the ELISA assay is a competitive ELISA

In some embodiments of the system, performing the plurality of instances of the assay comprises performing two or more of the plurality of instances at two or more times.

In some embodiments of the system, the two or more times constitute a time course of at least about one week.

In some embodiments of the system, performing the plurality of instances of the assay comprises performing two or more of the plurality of instances simultaneously.

In some embodiments of the system, the plurality of concentration data points comprises data points pertaining to a concentration a target analyte.

In some embodiments of the system, the plurality of concentration data points comprises data points pertaining to a concentration of a control.

In some embodiments of the system, the control is a negative control, non-specific binding control, blank control, detection antibody control, negative matrix control, or positive control.

In some embodiments of the system, the plurality of concentration data points comprises data points pertaining to a solution concentration.

In some embodiments of the system, the plurality of concentration data points comprises data points pertaining to an absolute amount of a target analyte.

In some embodiments of the system, the plurality of concentration data points comprises data points pertaining to a measurement associated with concentration.

In some embodiments of the system, the measurement associated with concentration is an optical density (OD) measurement.

In some embodiments of the system, the plurality of concentration data points comprises data points pertaining to a mean, lowest standard deviation mean, highest standard deviation mean, or middle control concentration.

In some embodiments of the system, the significant change point reflects inter-assay variability.

In some embodiments of the system, the significant change point reflects intra-assay variability.

In some embodiments of the system, two or more concentration data points of the plurality of concentration data points are in the same format.

In some embodiments of the system, the one or more assay parameters correlated with the significant change point comprise an environmental factor.

In some embodiments of the system, the environmental factor comprises a temperature, humidity, light, or contaminant.

In some embodiments of the system, the one or more assay parameters correlated with significant change points comprise an equipment factor.

In some embodiments of the system, the equipment factor comprises a reagent change, reagent aging, reagent expiration, reagent contamination, reagent failure, hardware change, hardware aging, hardware contamination, hardware failure, instrument change, instrument failure, or instrument calibration.

In some embodiments of the system, the one or more assay parameters correlated with the significant change point comprise a human factor associated with one or more humans who performed or assisted in performing the assay.

In some embodiments of the system, the human factor comprises performance variability, performance error, or operator change.

In some embodiments of the system, identifying the significant change point comprises determining an expected change point population in the plurality of concentration data points.

In some embodiments of the system, identifying the significant change point comprises selecting a first segment of concentration data points of the plurality of concentration data points, determining a first median value associated with the first segment of concentration data points, selecting a second segment of concentration data points of the plurality of concentration data points, wherein the concentration data points in the second segment are consecutive to the concentration data points in the first segment, determining a second median value associated with the second segment of concentration data points, comparing the first median value to the second median value and determining, based on the comparison between the first median value and the second median value, whether a candidate change point is located between the first segment and the second segment.

In some embodiments of the system, the first segment and the second segment comprise at least a threshold number of concentration data points.

In some embodiments of the system, the one or more processors are configured to receive the threshold number of concentration data points from a user.

In some embodiments of the system, the threshold number of concentration data points is determined based on the assay.

In some embodiments of the system, the one or more processors are configured to, for the first segment and the second segment, generate one or more mean values, generate one or more data point clusters, wherein each data point cluster is associated with a mean value of the one or more mean values and comprises concentration data points in the segment that are closest to the associated mean value, update the mean value for each data point cluster of the one or more data point clusters, wherein updating the mean value for a data point cluster comprises identifying a centroid of the data point cluster, and iteratively repeat the steps of generating one or more data point clusters and updating the mean values for each data point cluster until the mean values for each data point cluster no longer change.

In some embodiments of the system, the one or more processors are configured to generate one or more data point clusters within each of the first segment and the second segment, wherein the concentration data points in each data point cluster are normally distributed and have a unique mean value and a unique standard deviation value.

In some embodiments of the system, the one or more processors are configured to identify, for the first segment and the second segment, a principal data point cluster of the one or more data point clusters, wherein the principal data point cluster comprises at least a threshold percentage of a total number of concentration data points in the segment.

In some embodiments of the system, the one or more processors are configured to determine, for the candidate change point, a divergence value, wherein the divergence value measures a statistical difference between the principal data point cluster in the first segment and the principal data point cluster in the second segment.

In some embodiments of the system, the divergence value is a Jensen-Shannon divergence.

In some embodiments of the system, the one or more processors are configured to determine, for the candidate change point, a median change value, wherein the median change value measures a difference between the first median value associated with the first segment and the second median value associated with the second segment.

In some embodiments of the system, the one or more processors are configured to determine whether the one or more statistical characteristics of the plurality of concentration data points change by more than a threshold value by determining a weighted combination of the divergence value and the median change value.

In some embodiments of the system, the weighted combination of the divergence value and the median change value is characterized by a weight parameter.

In some embodiments of the system, the one or more processors are configured to receive the weight parameter from a user.

In some embodiments of the system, the weight parameter is determined based on the assay.

In some embodiments of the system, the one or more processors are configured to delete one or more concentration data points of the plurality of concentration data points that correspond to instances of the plurality of instances of performing the assay that occurred after the identified significant change point.

An example of a non-transitory computer readable storage medium may store instructions for determining a cause of a significant statistical change in a plurality of concentration data points obtained from an assay, wherein the instructions are configured to be executed by one or more processors of an electronic device to cause the device to receive assay information comprising the plurality of concentration data points obtained by performing a plurality of instances of the assay and a plurality of assay parameters, wherein each assay parameter of the plurality of assay parameters is associated with an instance of the plurality of instances of performing the assay, identify a significant change point that corresponds to a location in the plurality of concentration data points at which one or more statistical characteristics of the plurality of concentration data points change by more than a threshold value, correlate one or more assay parameters of the plurality of assay parameters with the identified significant change point by identifying an instance of the plurality of instances of performing the assay that corresponds to the location of the significant change point in the plurality of concentration data points, and determine the cause for the change in the one or more statistical characteristics of the plurality of concentration data points at the identified significant change point based on the correlation between the one or more assay parameters and the significant change point.

In some embodiments of the non-transitory computer readable storage medium, the assay is configured to measure a concentration of an analyte in a sample.

In some embodiments of the non-transitory computer readable storage medium, the analyte is a therapeutic analyte.

In some embodiments of the non-transitory computer readable storage medium, the analyte is a therapeutic polypeptide.

In some embodiments of the non-transitory computer readable storage medium, the analyte is an antibody or a fragment thereof.

In some embodiments of the non-transitory computer readable storage medium, the sample is a cell culture sample or a derivative thereof.

In some embodiments of the non-transitory computer readable storage medium, the assay is an immunoassay.

In some embodiments of the non-transitory computer readable storage medium, the assay is a competitive assay.

In some embodiments of the non-transitory computer readable storage medium, the assay is a non-competitive assay.

In some embodiments of the non-transitory computer readable storage medium, the assay is a non-homogenous assay.

In some embodiments of the non-transitory computer readable storage medium, the assay is a homogenous assay.

In some embodiments of the non-transitory computer readable storage medium, the assay is an ELISA assay.

In some embodiments of the non-transitory computer readable storage medium, the ELISA assay is a direct ELISA assay.

In some embodiments of the non-transitory computer readable storage medium, the ELISA assay is a sandwich ELISA assay.

In some embodiments of the non-transitory computer readable storage medium, the ELISA assay is a competitive ELISA assay.

In some embodiments of the non-transitory computer readable storage medium, performing the plurality of instances of the assay comprises performing two or more of the plurality of instances at two or more times.

In some embodiments of the non-transitory computer readable storage medium, the two or more times constitute a time course of at least about one week.

In some embodiments of the non-transitory computer readable storage medium, performing the plurality of instances of the assay comprises performing two or more of the plurality of instances simultaneously.

In some embodiments of the non-transitory computer readable storage medium, the plurality of concentration data points comprises data points pertaining to a concentration a target analyte.

In some embodiments of the non-transitory computer readable storage medium, the plurality of concentration data points comprises data points pertaining to a concentration of a control.

In some embodiments of the non-transitory computer readable storage medium, the control is a negative control, non-specific binding control, blank control, detection antibody control, negative matrix control, or positive control.

In some embodiments of the non-transitory computer readable storage medium, the plurality of concentration data points comprises data points pertaining to a solution concentration.

In some embodiments of the non-transitory computer readable storage medium, the plurality of concentration data points comprises data points pertaining to an absolute amount of a target analyte.

In some embodiments of the non-transitory computer readable storage medium, the plurality of concentration data points comprises data points pertaining to a measurement associated with concentration.

In some embodiments of the non-transitory computer readable storage medium, the measurement associated with concentration is an optical density (OD) measurement.

In some embodiments of the non-transitory computer readable storage medium, the plurality of concentration data points comprises data points pertaining to a mean, lowest standard deviation mean, highest standard deviation mean, or middle control concentration.

In some embodiments of the non-transitory computer readable storage medium, the significant change point reflects inter-assay variability.

In some embodiments of the non-transitory computer readable storage medium, the significant change point reflects intra-assay variability.

In some embodiments of the non-transitory computer readable storage medium, two or more concentration data points of the plurality of concentration data points are in the same format.

In some embodiments of the non-transitory computer readable storage medium, the one or more assay parameters correlated with the significant change point comprise an environmental factor.

In some embodiments of the non-transitory computer readable storage medium, the environmental factor comprises a temperature, humidity, light, or contaminant.

In some embodiments of the non-transitory computer readable storage medium, the one or more assay parameters correlated with significant change points comprise an equipment factor.

In some embodiments of the non-transitory computer readable storage medium, the equipment factor comprises a reagent change, reagent aging, reagent expiration, reagent contamination, reagent failure, hardware change, hardware aging, hardware contamination, hardware failure, instrument change, instrument failure, or instrument calibration.

In some embodiments of the non-transitory computer readable storage medium, the one or more assay parameters correlated with the significant change point comprise a human factor associated with one or more humans who performed or assisted in performing the assay.

In some embodiments of the non-transitory computer readable storage medium, the human factor comprises performance variability, performance error, or operator change.

In some embodiments of the non-transitory computer readable storage medium, identifying the significant change point comprises determining an expected change point population in the plurality of concentration data points.

In some embodiments of the non-transitory computer readable storage medium, identifying the significant change point comprises selecting a first segment of concentration data points of the plurality of concentration data points, determining a first median value associated with the first segment of concentration data points, selecting a second segment of concentration data points of the plurality of concentration data points, wherein the concentration data points in the second segment are consecutive to the concentration data points in the first segment, determining a second median value associated with the second segment of concentration data points, comparing the first median value to the second median value, and determining, based on the comparison between the first median value and the second median value, whether a candidate change point is located between the first segment and the second segment.

In some embodiments of the non-transitory computer readable storage medium, the first segment and the second segment comprise at least a threshold number of concentration data points.

In some embodiments of the non-transitory computer readable storage medium, the instructions, when executed by the one or more processors of the electronic device, are configured to cause the electronic device to receive the threshold number of concentration data points from a user.

In some embodiments of the non-transitory computer readable storage medium, the threshold number of concentration data points is determined based on the assay.

In some embodiments of the non-transitory computer readable storage medium, the instructions, when executed by the one or more processors of the electronic device, are configured to cause the device to, for the first segment and the second segment, generate one or more mean values, generate one or more data point clusters, wherein each data point cluster is associated with a mean value of the one or more mean values and comprises concentration data points in the segment that are closest to the associated mean value, update the mean value for each data point cluster of the one or more data point clusters, wherein updating the mean value for a data point cluster comprises identifying a centroid of the data point cluster, and iteratively repeat the steps of generating one or more data point clusters and updating the mean values for each data point cluster until the mean values for each data point cluster no longer change.

In some embodiments of the non-transitory computer readable storage medium, the instructions, when executed by the one or more processors of the electronic device, are configured to cause the device to generate one or more data point clusters within each of the first segment and the second segment, wherein the concentration data points in each data point cluster are normally distributed and have a unique mean value and a unique standard deviation value.

In some embodiments of the non-transitory computer readable storage medium, the instructions, when executed by the one or more processors of the electronic device, are configured to cause the device to identify, for the first segment and the second segment, a principal data point cluster of the one or more data point clusters, wherein the principal data point cluster comprises at least a threshold percentage of a total number of concentration data points in the segment.

In some embodiments of the non-transitory computer readable storage medium, the instructions, when executed by the one or more processors of the electronic device, are configured to cause the device to determine, for the candidate change point, a divergence value, wherein the divergence value measures a statistical difference between the principal data point cluster in the first segment and the principal data point cluster in the second segment.

In some embodiments of the non-transitory computer readable storage medium, the divergence value is a Jensen-Shannon divergence.

In some embodiments of the non-transitory computer readable storage medium, the instructions, when executed by the one or more processors of the electronic device, are configured to cause the device to determine, for the candidate change point, a median change value, wherein the median change value measures a difference between the first median value associated with the first segment and the second median value associated with the second segment.

In some embodiments of the non-transitory computer readable storage medium, the instructions, when executed by the one or more processors of the electronic device, are configured to cause the device to determine whether the one or more statistical characteristics of the plurality of concentration data points change by more than a threshold value by determining a weighted combination of the divergence value and the median change value.

In some embodiments of the non-transitory computer readable storage medium, the weighted combination of the divergence value and the median change value is characterized by a weight parameter.

In some embodiments of the non-transitory computer readable storage medium, the instructions, when executed by the one or more processors of the electronic device, are configured to cause the device to receive the weight parameter from a user.

In some embodiments of the non-transitory computer readable storage medium, the weight parameter is determined based on the assay.

In some embodiments of the non-transitory computer readable storage medium, the instructions, when executed by the one or more processors of the electronic device, are configured to cause the device to delete one or more concentration data points of the plurality of concentration data points that correspond to instances of the plurality of instances of performing the assay that occurred after the identified significant change point.

### BRIEF DESCRIPTION OF THE FIGURES

The following figures show various systems and methods for identifying and validating change points in concentration data obtained from an assay. The systems and methods shown in the figures may, in some embodiments, have any one or more of the characteristics described herein.

FIGS. 1A-1C show schematic representations of ELISA assay formats for obtaining concentration data of an analyte. FIG. 1A shows a direct ELISA format. FIG. 1B shows a sandwich ELISA format. FIG. 1C shows a competitive ELISA format.

FIG. 2 shows an example of concentration data points obtained from an assay.

FIG. 3 shows an example of deviations in concentration data obtained from an assay.

FIG. 4 shows examples of change points in a data set.

FIG. 5 shows a method for determining a cause of a significant statistical change in a plurality of concentration data points obtained from an assay.

FIG. 6 shows a method for identifying candidate change points using binary segmentation.

FIG. 7 shows change points in a signal that have been identified using binary segmentation.

FIGS. 8A-8C show methods for validating the significance of candidate change points. FIG. 8A shows a method for generating data point clusters using a k-means algorithm. FIG. 8B shows a method for generating data point clusters using Gaussian mixture models. FIG. 8C shows a method for determining whether statistical characteristics of the plurality of concentration data points surrounding a candidate change point change by more than a threshold value.

FIGS. 9A-9B shows example segments and example data point clusters in concentration data.

FIGS. 10A-10C show example contour plots of a function that can be used to generate loss values for candidate change points.

FIG. 11 shows methods for proceeding after the cause of a significant statistical change in a plurality of concentration data points obtained from an assay has been identified.

FIG. 12 shows a system for determining a cause of a significant statistical change in a plurality of concentration data points obtained from an assay.

FIG. 13 illustrates an example of a computing system.

FIGS. 14A-14J show various examples of significant change points that have been identified in a plurality of concentration data points using the disclosed methods.

### DETAILED DESCRIPTION

The following disclosure describes methods for identifying and determining causes for statistical changes in process data by determining locations in the data where significant changes in the data have occurred. These locations, known as “change points”, are typically points in time where statistical shifts have occurred in a time series of data. The methods provided can allow laboratory analysts to correlate change points in process data with changes in measurement parameters that may have occurred at the times of the identified change points. This may enable the root causes of the changes in the process data to be efficiently extracted even when ground truth data for the process is limited or unavailable.

In the disclosure, the methods are explained in the context of assays, i.e., investigative processes that can be used to assess the presence of an analyte such as a drug, a cell, or a chemical substance. This context is not intended to limit the disclosure; the provided methods can be used to evaluate any data set that contains a plurality of repeated measurements.

Obtaining Concentration Data from Assays

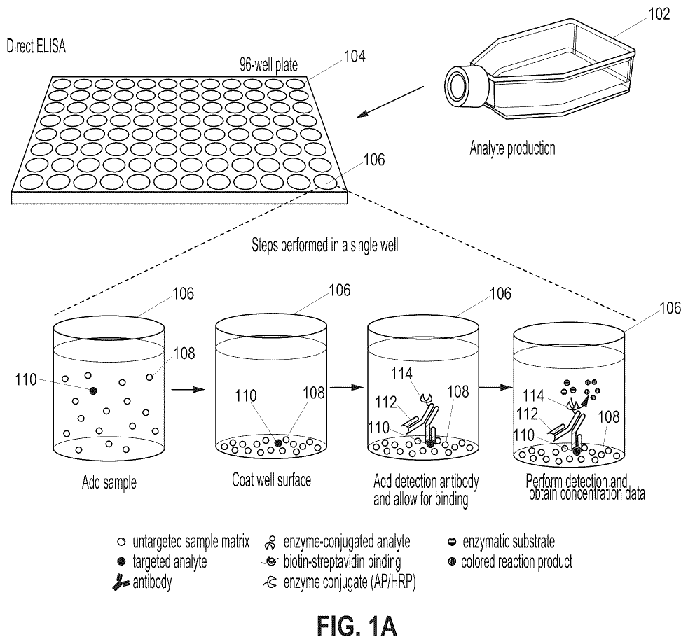

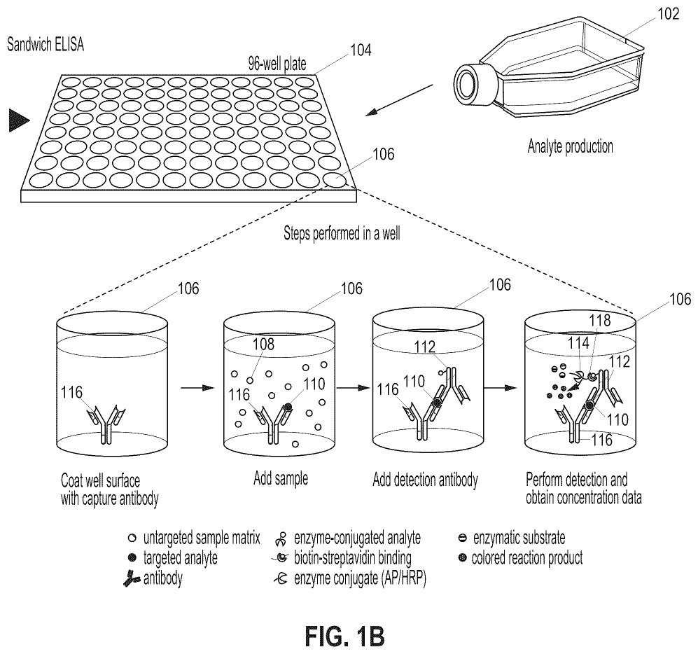

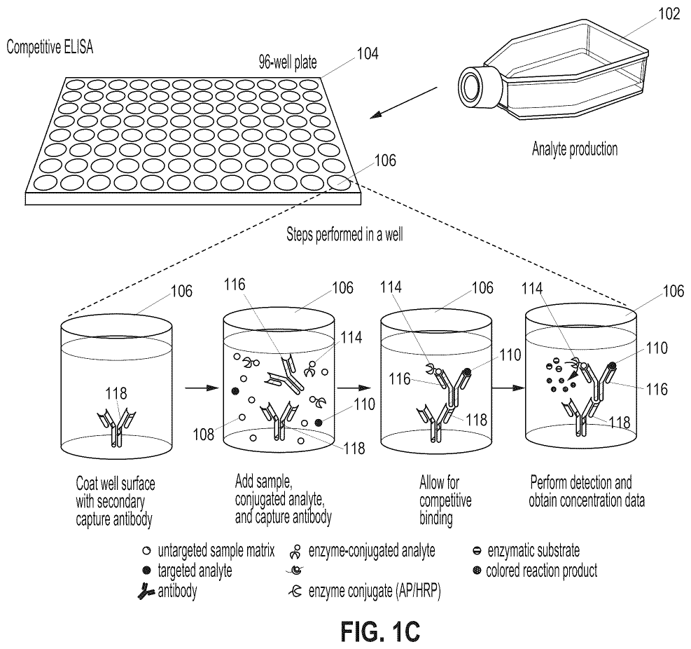

An assay is a process that can be used to determine the presence of an analyte. There are numerous assay types and formats that are well known in the art. For example, as shown in FIGS. 1A-1C, various enzyme-linked immunosorbent assay (ELISA) formats are available, including a direct ELISA format (FIG. 1A), a sandwich ELISA format (FIG. 1B), and a competitive ELISA format (FIG. 1C).

In the direct ELISA format (FIG. 1A), the sample 108 is coated onto a solid phase, such as a well 106 of a plate 104, and the desired antigen can be detected by antibodies 112. Negative and positive controls are available for the direct ELISA format. Such controls include, wavelength correction, blank controls (e.g., a dry well or a well contain ELISA buffer), S0 negative control (no standard or any form of analyte, e.g., sample, added to well), negative matrix control, and positive controls, such as B0 (to assess maximum color development). In some embodiments, standard curves can also be utilized for quantification purposes.

In the sandwich ELISA format (FIG. 1B), a capture antibody 116 is coated onto a solid phase, such as a well 106 of a plate 104. The capture antibody 116 specifically binds to the analyte of interest 110. Then, the sample 108 is added to the well 106 to allow for the capture antibody 120 to bind analyte, if present. Following this, a detection antibody 112 is added to the well to allow for analyte detection. Negative and positive controls are available for the sandwich ELISA format. Such controls include, wavelength correction, blank controls (e.g., a dry well or a well contain ELISA buffer), S0 negative control (no standard or any form of analyte, e.g., sample, added to well), negative matrix control, and positive controls, such as B0 (to assess maximum color development). In some embodiments, standard curves can also be utilized for quantification purposes.

In the competitive ELISA format (FIG. 1C), a secondary capture antibody 118 is coated onto a solid phase, such as a well 106 of a plate 104. The secondary capture antibody 118 specifically binds to a capture antibody 116, and the capture antibody 118 specifically binds the analyte of interest 110. To the well 106 containing the secondary capture antibody 118, the sample 108, a conjugated analyte 114, and a capture antibody 116 are added and competitive binding occurs. The more analyte 110 contained in the same, the less the conjugated analyte 114 will remain bound to the capture antibody 116. Detection can then proceed to collect concentration data. Negative and positive controls are available for the competitive ELISA format. Such controls include, wavelength correction, blank controls (e.g., a dry well or a well contain ELISA buffer), non-specific binding (NSB) control, negative matrix control, and positive controls, such as B0 (to assess maximum color development). In some embodiments, standard curves can also be utilized for quantification purposes.

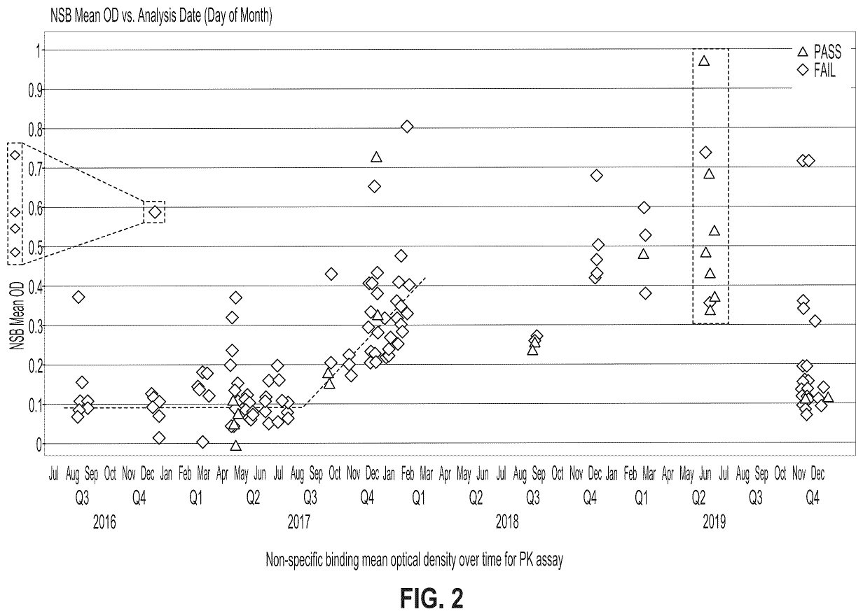

Concentration data, such as concentration data of an antibody produced from a cell culture, can be obtained using an assay such as those shown in FIGS. 1A-1C. Concentration data may include a plurality of concentration data points that may have been collected over a period of time. In some situations, concentration data may comprise data that directly pertains to the concentration of an analyte, a control, or a solution. In other scenarios, concentration data may include data that pertains to measurements associated with the concentration of an analyte, a control, or a solution. For instance, as shown in FIG. 2, concentration data may be a time series of measurements of mean optical density.

Many parameters may affect the data obtained from an assay. These assay parameters may be related to the environment in which the assay is performed, the equipment used to perform the assay, or the humans involved with performing the assay. Ideally, assay parameters remain unchanged over the entire period of time in which an assay is used to obtain concentration data. However, when numerous concentration data points are obtained from an assay over an extended period of time (e.g., over several weeks or several months), changes to some assay parameters may be unavoidable. In some cases, these changes may cause significant fluctuations in the concentration data.

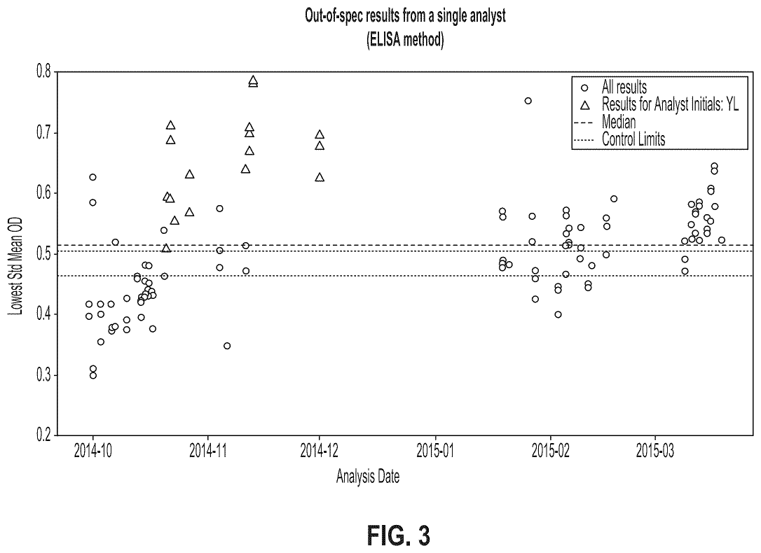

FIG. 3 shows an example of deviations in concentration data obtained from an assay caused by a change in an assay parameter-specifically, a change in laboratory analyst. As shown, concentration data obtained by one of the analysts during the time period between mid-October 2014 and December 2014 lies significantly outside of the range of the rest of the concentration data. Such outlying data may be suggestive of potential systemic error associated with the assay parameter (in this case, error systematically propagated by the analyst who collected the outlying data).

Ensuring that the concentration data obtained from an assay is accurate may require changes in the concentration data to be identified and the root causes of the changes to be evaluated. Often, changes in concentration data may be the result of changes in an assay parameter, as illustrated by the example concentration data shown in FIG. 3. In other cases, changes in concentration data may be the result of processes or reactions that should be further investigated. Regardless of the root causes, the efficient location and validation of the significance of change points in concentration data may an essential step in the analysis of the concentration data.

### Method Overview

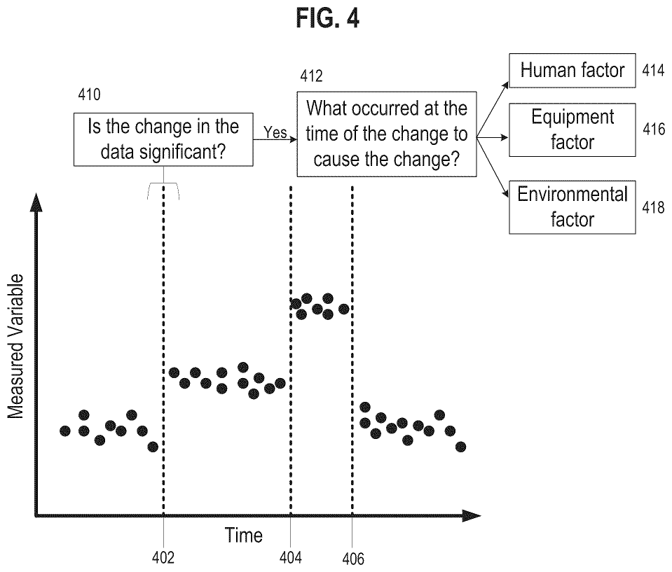

The methods described can identify change points in a plurality of concentration data points obtained from an assay. As previously discussed, a change point may be a point at which a statistical change occurs in the concentration data. Examples of change points in a data set are illustrated in FIG. 4.

Changes in the measured variable are reflected in the data shown in FIG. 4 at a first point in time 402, at a second point in time 404, and at a third point in time 406. After each of the points in time 402-406, statistical characteristics (e.g., median, mean, etc.) of the data appear to change. The methods provided herein can be used to algorithmically identify such change points in a data set such as a data set comprising a plurality of concentration data points obtained from an assay. After the locations of change points in a data set have been identified, the methods can validate the significance of each change point (block 410). In other words, the methods can determine whether the differences between the statistical distribution of the plurality of data points before a change point and the statistical distribution of the plurality of data points after a change point are large enough to indicate that errors or other events worthy of further investigation occurred at the time of the change point. If it is determined that a change point is significant, then personnel associated with the data (e.g., laboratory analysts who collected the data) may be notified so that the root causes of the changes in the data can be evaluated (block 412). The root causes of the changes in the data may include one or more of a human factor (block 414), an equipment factor (block 416), an environmental factor (block 418), etc.

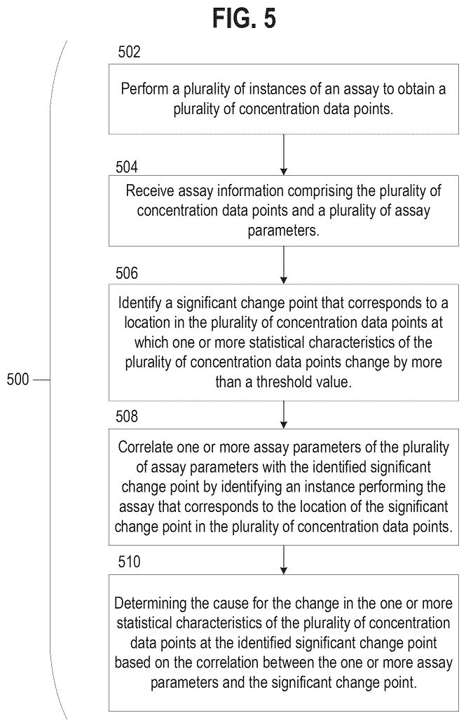

FIG. 5 shows an example of a method 500 for determining a cause of a significant statistical change in concentration data obtained from an assay. In some embodiments, one or more steps of method 500 may be executed by one or more processors in a system configured to identify and validate change points in a data set (e.g., by processors in a computer belonging to laboratory analyst who helped perform the assay).

As shown, method 500 may include a first step 502, wherein a plurality of instances of an assay (or, in some embodiments, a plurality of instances of multiple assays) may be performed to obtain a plurality of concentration data points. The assay performed may be of any type or format known in the art. For example, the assay may be an immunoassay, a competitive assay, a non-competitive assay, a non-homogenous assay, or a homogenous assay. Optionally, the assay may be an ELISA assay such as a direct ELISA assay, a sandwich ELISA assay, or a competitive ELISA assay (see FIGS. 1A-1C for schematic representations of ELISA assays).

The assay may be configured to measure the concentration of an analyte in a sample. Optionally, the analyte may be a therapeutic analyte, a therapeutic polypeptide, an antibody, or a fragment of an antibody. The sample may be a cell culture sample or a derivative thereof.

In some embodiments, performing the plurality of instances of the assay may comprise performing two or more of the plurality of instances at two or more times. The two or more times may constitute a time course of at least about one day, at least about one week, at least about one month, at least about six months, at least about one year, or at least about five years. In other words, two or more of the plurality of concentration data points obtained in step 502 may be obtained at two or more different times in a single day, two or more different days in a week, two or more different days in a month, or two or more different days in a year. In some embodiments, performing the plurality of instances of the assay may comprise performing two or more of the plurality of instances in parallel (i.e., simultaneously).

The plurality of concentration data points may comprise data points pertaining to a concentration of a target analyte, an absolute amount of a target analyte, a concentration of a control (e.g., a negative control, a non-specific binding control, a blank control, a detection antibody control, a negative matrix control, or a positive control), and/or a solution concentration. In some aspects of method 500, the plurality of concentration data points may include data points pertaining to a measurement associated with concentration, such as an optical density (OD) measurement. Optionally, the plurality of concentration data points may comprise data points pertaining to a mean, low reference sample, medium reference sample, high reference sample, low control, medium control, high control. In some embodiments, two or more of the plurality of concentration data points may be in the same format or in different formats.

After the plurality of concentration data points have been obtained from an assay in step 502, method 500 may proceed to a step 504, wherein assay information may be received. The assay information may include the plurality of concentration data points as well a plurality of assay parameters. Assay parameter information may be recorded during each instance of performing the assay; each assay parameter of the plurality of assay parameters may be associated with an instance of the plurality of instances of performing the assay and may indicate a characteristic or property of the assay or a characteristic or property of a factor associated with the assay during that instance. For example, assay parameters associated with an instance of performing the assay may comprise environmental factors associated with the environment in which assay was performed (e.g., temperature, humidity, light, or the presence of one or more contaminants), equipment factors associated with the equipment used to perform the assay (e.g., reagent changes, reagent aging, reagent expiration, reagent contamination, reagent failure, hardware change, hardware aging, hardware contamination, hardware failure, instrument change, instrument failure, or instrument calibration), or human factors associated with one or more operators who performed or assisted in performing the assay (e.g., performance variability among operators, performance error by one or more of the operators, or operator changes). In some embodiments, multiple assay parameters of the plurality of assay parameters may be associated with a single instance of performing the assay.

After the assay information is received in step 504, method 500 may proceed to a step 506, wherein a significant change point may be identified. The significant change point may correspond to a location in the plurality of concentration data points at which one or more statistical characteristics of the plurality of concentration data points change by more than a threshold value. A significant change point may be a location among the plurality of concentration data points at which a median, a mean, a variance, and/or a correlation of the plurality of concentration data points has changed or at which an anomaly in the plurality of concentration data points has been identified.

In order to identify the significant change point, one or more candidate change points may first be identified. The validity of each candidate change point may then be determined by quantifying the statistical changes in the concentration data points before and after each candidate change point. If the statistical changes in the concentration data points before and after a candidate change point are determined to be significant (e.g., if the quantification of the changes exceeds a cutoff value), then that candidate change point may be identified as a significant change point.

Once a significant change point has been identified in step 506, method 500 may move to a step 508, wherein one or more assay parameters of the plurality of assay parameters may be correlated with the identified significant change point. In some embodiments, one or more assay parameters may be correlated with the significant change point by identifying an instance of the plurality of instances of performing the assay that corresponds to the location of the significant change point in the plurality of concentration data points. For example, a significant change point may identified at a location in the plurality of concentration data points that corresponds to a date (e.g., a day, month, and year). The instance of performing the assay may be identified based on this date. After the instance of performing the assay is identified, one or more assay parameters associated with the instance may be correlated with the significant change point.

After one or more assay parameters are correlated with the identified significant change point in step 508, method 500 may proceed to a step 510, wherein a cause for the change in the one or more statistical characteristics of the plurality of concentration data points at the significant change point may be determined based on the correlation between the one or more assay parameters and the significant change point. If, for example, the one or more assay parameters indicate that a change in operator occurred at or around the time of the significant change point, determining the cause for the change in the one or more statistical characteristics of the plurality of concentration data points may comprise determining that the operator who performed the assay before the significant change point or the operator who performed the assay after the significant change point may have made an error. Once the potential source of the change is determined, the root causes of the changes may be efficiently and accurately verified.

The following sections provide additional description of the step of identifying a significant change point (step 506 of method 500)—specifically, description of how candidate change points may be identified and how the significance of candidate change points may be validated.

### Identifying Candidate Change Points

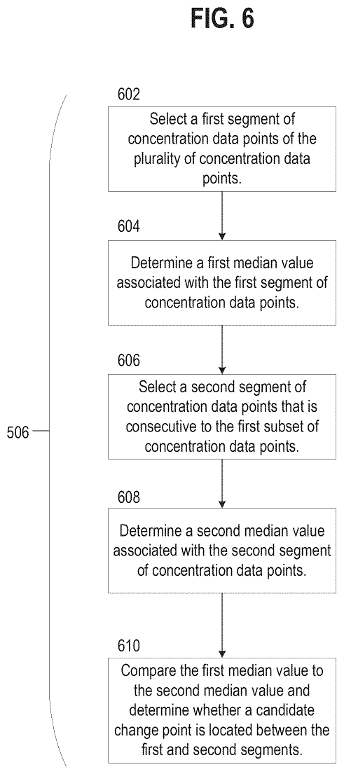

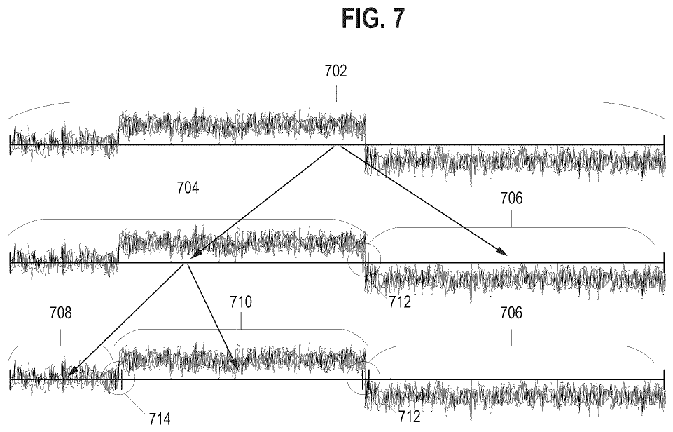

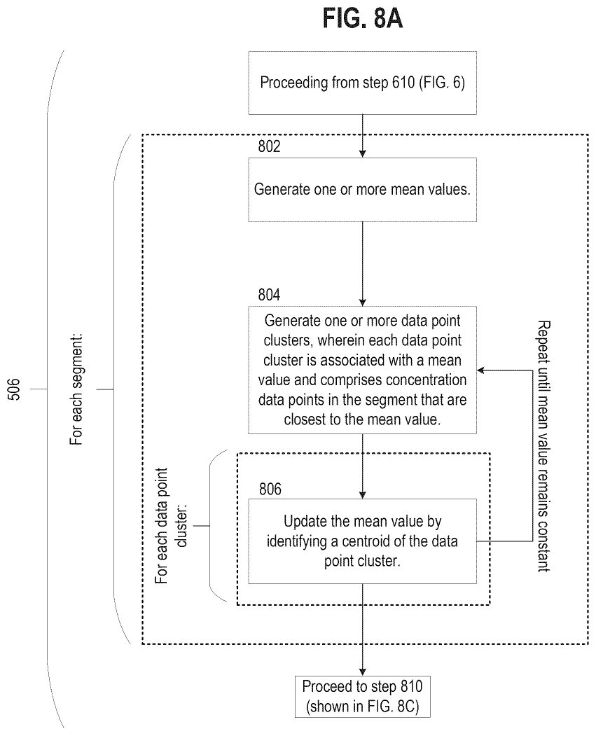

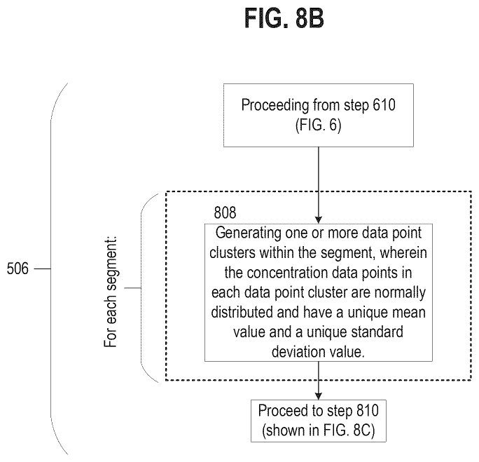

After the plurality of concentration data points are obtained by performing a plurality of instances of an assay (step 502 of method 500), a significant change point may be identified (step 506 of method 500). As described in the preceding section, identifying a significant change point may involve identifying one or more candidate change points. FIG. 6 shows an example of a method for identifying one or more candidate change points using a binary segmentation algorithm.

The binary segmentation algorithm begins by selecting a first segment of concentration data points of the plurality of concentration data points (step 602). In some embodiments, the first segment of concentration data points may comprise at least a threshold number (N) of concentration data points. The threshold number of concentration data points may help to ensure that only change points with a high likelihood of being significant are identified. In other words, the threshold number of concentration data points may reduce the sensitivity of the binary segmentation algorithm to small, random fluctuations in the plurality of concentration data points that are not indicative of significant changes. The threshold number of concentration data points may be provided by a user. Optionally, the threshold number of concentration data points may be determined based on the assay used to obtain the plurality of concentration data points. A first median value (m₁) associated with the first segment of concentration data points may be determined (step 604) after the first segment of concentration data points is selected.

Next, in a step 606, a second segment of concentration data points of the plurality of concentration data points is selected. The concentration data points in the second segment may be consecutive to the concentration data points in the first segment. Like the first segment, the second segment of concentration data points may comprise at least the threshold number (N) of concentration data points in order to reduce the sensitivity of the binary segmentation algorithm to unimportant fluctuations. After the second segment of concentration data points is selected, a second median value (m₂) associated with the second segment of concentration data points may be determined (step 608).

Once the first median value and the second median value have been determined, the first median value may be compared to the second median value to determine whether a candidate change point is located between the first and second segments of concentration data points (step 610). Comparing the first median value to the second median value may involve determining whether the difference between the first median value and the second mean value (e.g., Δ=|m₁−m₂|) exceeds the coefficient of variation of the first segment (CV₁) scaled by a scaling parameter (k), where the coefficient of variation of the first segment is the ratio of the standard deviation of the first segment (σ₁) to the mean of the first segment (μ₁). The scaling parameter (k) may be provided by a user and may depend on the assay performed to obtain the plurality of concentration data points. The scaling parameter may be about 0.1, about 0.2, about 0.3, about 0.4, about 0.5, about 0.6, about 0.7, about 0.8, or about 0.9. In some embodiments, the scaling parameter may be greater than 0.1, greater than 0.2, greater than 0.3, greater than 0.4, greater than 0.5, greater than 0.6, greater than 0.7, greater than 0.8, or greater than 0.9. In some embodiments, the scaling parameter may be less than 0.1, less than 0.2, less than 0.3, less than 0.4, less than 0.5, less than 0.6, less than 0.7, less than 0.8, or less than 0.9.

If a candidate change point is identified between the first and second segments in step 610, steps 602-610 may be repeated for the concentration data points in the first segment and the concentration data points in the second segment in order to determine whether additional candidate change points lie within the first segment and/or the second segment. Several iterations of a binary segmentation method are illustrated in FIG. 7. As shown, an entire signal 702 (e.g., the plurality of concentration data points obtained from the assay) is divided into a first segment of data points 704 and a second segment of data points 706, as described in step 602 shown in FIG. 6. A candidate change point 712 is identified between the first segment 704 and the second segment 706, as described in steps 604-610 of FIG. 6. The binary segmentation process is then repeated for the data points in the first segment 704. In other words, the first segment 704 is divided into a two segments of data points 708 and 710, and a second candidate change point 714 is identified between segments 708 and 710.

The binary segmentation process may continue to iterate until one or more cessation conditions are met, for example until a maximum number of iterations have been performed or until a threshold number of candidate change points have been identified. Once the cessation conditions are met, the binary segmentation process may cease.

### Validating the Significance of Candidate Change Points

Once one or more candidate change points have been identified in the plurality of concentration data points (e.g., using the binary segmentation technique described in FIGS. 6-7), the validity of each candidate change point may be determined. In some embodiments, validating the significance of candidate change points may require data point clusters (e.g., statistical patterns) to be identified in segments of concentration data points before and after each candidate change point.

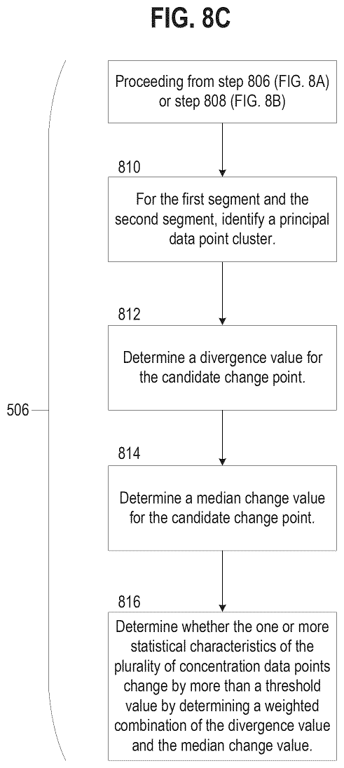

There are many possible methods for generating such data point clusters. FIG. 8A describes a first method, which employs a k-means algorithm to generate data point clusters in the plurality of concentration data points. If a candidate change point is identified between a first segment of concentration data points and a second segment of concentration data points (e.g., as described in method 600 shown in FIG. 6), then for each segment, one or more mean values may be generated (step 802). In some embodiments, the mean values for a segment may be generated by randomly assigning one or more concentration data points within the segment to be the mean values. After the one or more mean values are generated, one or more data point clusters may be generated (step 804). Each of the one or more data point clusters may be associated with a mean value of the one or more mean values generated in step 802. The data point cluster associated with given mean value may comprise concentration data points in the respective segment that are closest to that mean value. The distance of a concentration data point to a mean value may be determined using a norm (e.g., a Euclidean norm). After the one or more data point clusters are generated in step 804, the mean value for each data point cluster may be updated by identifying the centroid (i.e., the concentration data point in the center) of the data point cluster and assigning said centroid to be the updated mean value. Steps 804-806 may be iteratively repeated for each segment until the mean values no longer change at the updating step.

FIG. 8B describes an alternative method for generating data point clusters that utilizes Gaussian mixture models. If a candidate change point is identified between a first segment of concentration data points and a second segment of concentration data points (e.g., as described in method 600 shown in FIG. 6), one or more data point clusters may be identified for each segment (step 808). In this case, each of the one or more data point clusters may be normally distributed and may have a unique mean value and a unique variance value. In other words, the one or more data point clusters in each segment may be generated by dividing the concentration data points in each segment into distinct, normally distributed groups of concentration data points.

In some embodiments, identifying data point clusters within segments of concentration data points (e.g., using the methods shown in FIGS. 8A-8B) may comprise determining an expected number of data point clusters within each segment. For example, techniques such as the Bayesian information criterion may be used to find the number of data point clusters within a segment of concentration data points.

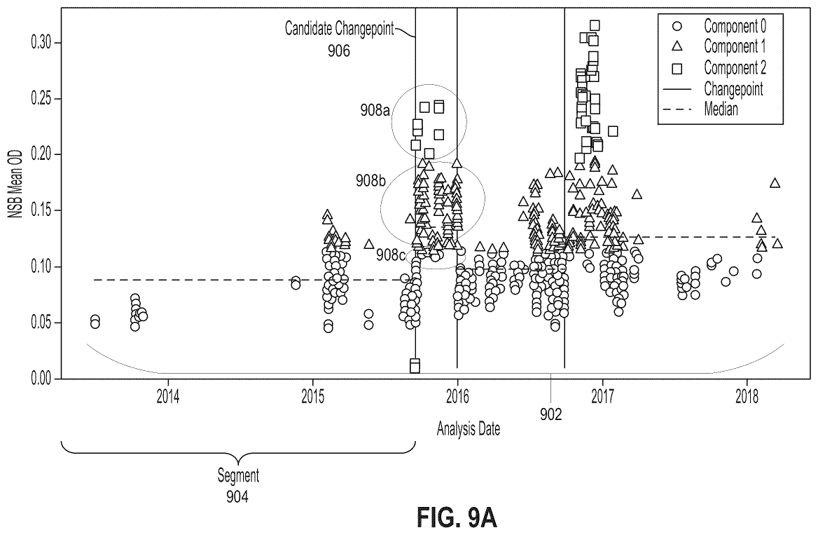

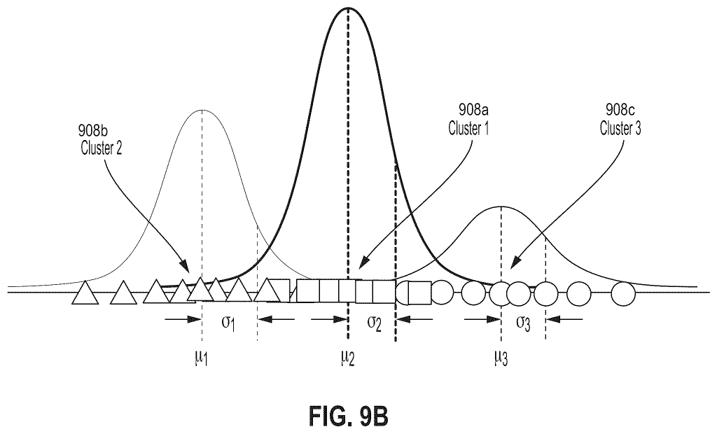

Example segments and example data point clusters in a plurality of concentration data points are provided in FIGS. 9A-9B. As shown in FIG. 9A, the plurality of concentration data points 902 may be divided into segments 904 separated by candidate change points 906. Within each segment, data point clusters 908a, 908b, and 908c can be generated. FIG. 9B illustrates how, when data point clusters 908a-c are generated using the method shown in FIG. 8B, each cluster may be uniquely normally distributed, i.e., may be normally distributed with a unique mean (μ) and a unique standard deviation (σ).

Once the one or more data point clusters in each segment of the plurality of concentration data points have been identified (e.g., by using the k-means algorithm shown in FIG. 8A, the Gaussian mixture model method shown in FIG. 8B), the significance of each candidate change points may be validated. Validating the significance of a candidate change point may involve determining whether the one or more statistical characteristics of the plurality of concentration data points change by more than a threshold value, as shown in FIG. 8C. First, for each segment of concentration data points, a principal data point cluster of the one or more data point clusters within the segment may be identified (step 810). The principal data point cluster for a segment may be a data point cluster that contains at least a threshold percentage of the concentration data points in the segment. For example, the principal data point cluster for a segment may be the data point cluster that contains at least 5%, at least 10%, at least 15%, at least 20%, at least 25%, or at least 30% of the concentration data points in the segment.

After the principal data point clusters for each segment are identified, a divergence value for each candidate change point may be determined (step 812). For a candidate change point located between a first segment and a second segment of concentration data points, the divergence value may measure a statistical difference between the principal data point cluster in the first segment and the principal data point cluster in the second segment. Specifically, the divergence value may measure a difference in the probability distribution of cluster membership between data point cluster membership in the first segment and data point cluster membership in the second segment. The divergence value for a candidate change point may be determined using a method for measuring similarities between probability distributions. The Jensen-Shannon divergence (JSD) is an example of such a method. If P is a probability distribution representing the principal data point cluster in the segment located just prior to a candidate change point (the first segment) and Q is a probability distribution representing the principal data point cluster in the segment that immediately follows the candidate change point (the second segment), then the JSD for the candidate change point is defined to be:

$$J S D ( P ‖ Q ) = \frac{1}{2} D ( P ‖ M ) + \frac{1}{2} D ( Q ‖ M )$$

$$where :$$

$$M = \frac{1}{2} ( P + Q )$$

and D(P∥M) and D(Q∥M) are the Kullback-Leibler divergences.

In addition to a divergence value, a median change value may be determined for each candidate change point (step 814). The median change value for a change point may measure a difference between a median value of the segment that is located just prior to the candidate change point (e.g., the first median value associated with the first segment, as described in step 604 of method 600 shown in FIG. 6) and a median value of the segment that immediately follows the candidate change point (e.g., the second median value associated with the second segment, as described in step 608 of method 600 shown in FIG. 6). In some embodiments, the median value of a segment may be the median of all of the concentration data points in the segment or the median of the concentration data points in the principal data point cluster in the segment.

The changes in the statistical characteristics before and after each candidate change point may then be quantified in order to determine whether the one or more statistical characteristics of the plurality of concentration data points change by more than a threshold value (step 816). Quantifying the changes in the statistical characteristics of the plurality of concentration data points before and after a candidate change point may comprise determining a loss value that comprises a weighted combination of the divergence value for the change point and the median change value for the change point. The weighted combination may be characterized by a weight parameter (λ). This weight parameter may be received from a user and may depend on the assay performed to obtain the plurality of concentration data points.

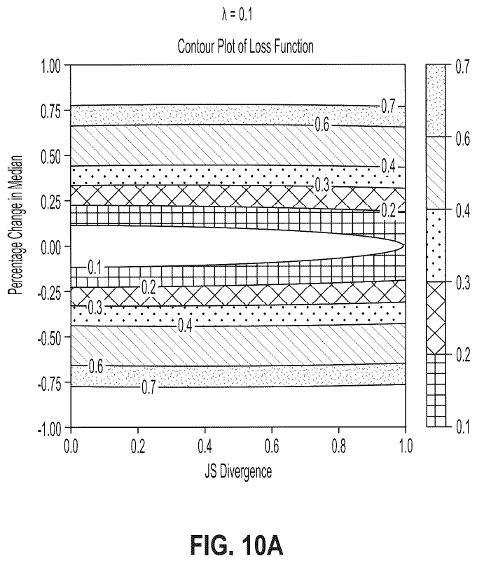

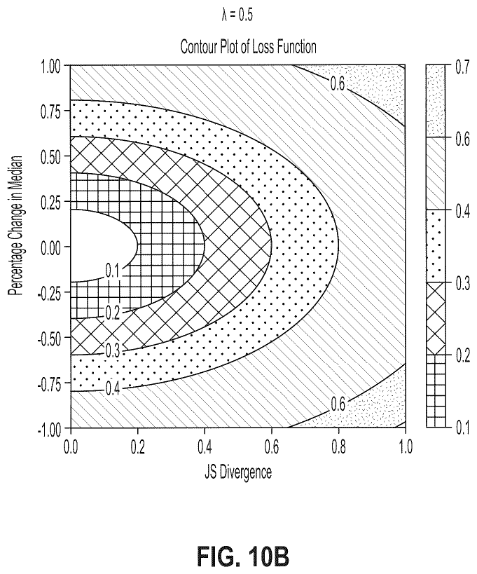

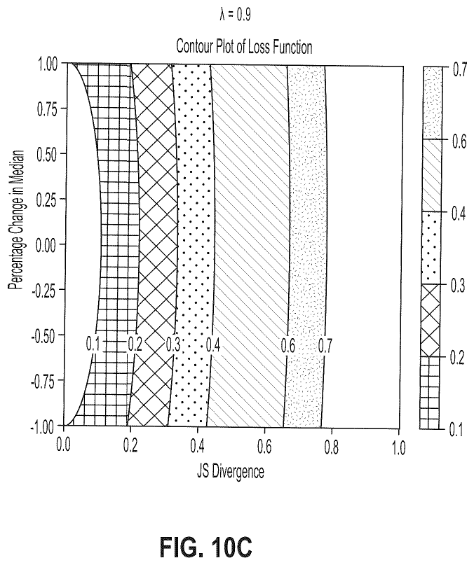

As previously described, the divergence value for a candidate change point may be determined using the Jensen-Shannon divergence (JSD). In this case, the loss value for a candidate change point may be defined as follows:

$$Loss Value = \sqrt{{{λ}^{2} {( tanh ( M C ) )}^{2} + {( 1 - λ )}^{2} J S D )}^{2}}$$

where MC is the median change value for the candidate change point and JSD is the divergence value for the candidate change point. The dependence of this function on the weight parameter (λ) is shown in the contour plots of the function provided in FIGS. 10A-10C.

A candidate change point may be identified as a significant change point if the loss value for the candidate change point exceeds a threshold value. The threshold value may be given by a cutoff parameter (ε). This cutoff parameter may characterize a change point detection sensitivity. The specific context of the assay (e.g., the motivations for performing the assay) may determine the types of changes in the plurality of concentration data points that users wish to analyze. For example, if a user only wishes to investigate major fluctuations in the plurality of concentration data points, the user may employ a large cutoff parameter. Alternatively, if the user wishes to investigate less obvious fluctuations in the plurality of concentration data points, the user may employ a smaller cutoff parameter.

### Determining the Cause(s) of the Changes

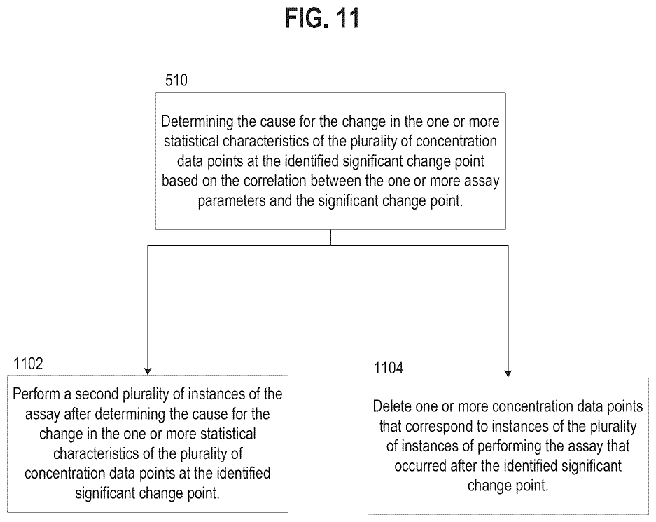

After a significant change point is identified (step 506 of method 500), the cause for the change in the one or more statistical characteristics of the plurality of concentration data points at the identified significant change point may be determined based on the correlation between the one or more assay parameters and the significant change point (step 510 of method 500). FIG. 11 shows optional methods for proceeding after the cause of a significant statistical change in a plurality of concentration data points obtained from an assay has been identified.

As shown, in some embodiments, a second plurality of instances of the assay after determining the cause for the change in the one or more statistical characteristics of the plurality of concentration data points at the identified significant change point (step 1102). The second plurality of instance of the assay may be performed with assay parameters that match the assay parameters associated with the instances of performing the assay that occurred prior to the identified significant change point or after the identified significant change point. In some embodiments, one or more concentration data points that correspond to instances of the plurality of instances of performing the assay that occurred before or after the identified significant change point may be removed from the plurality of concentration data points (step 1104). Step 1104 may be preferred in situations where the cause of the change is determined to be an error (e.g., a systemic error in performing the assay).

### System for Identifying and Validating Change Points

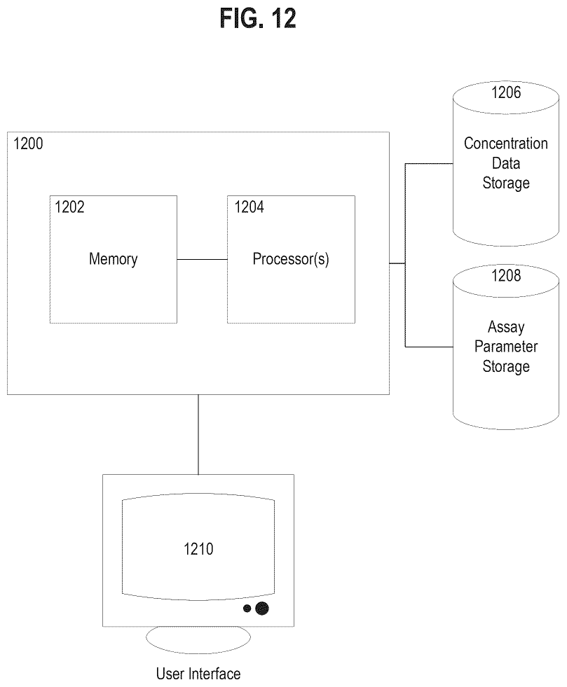

One or more steps of the described methods may be executed by a system or a device configured to identify and validate change points in a plurality of concentration data points obtained from an assay. An example of a system 1200 for determining a cause of a significant statistical change in a plurality of concentration data points obtained from an assay is shown in FIG. 12. System 1200 may comprise a memory 1202 coupled to one or more processors 1204. Memory 1202 may store instructions that, when executed by processor(s) 1204, cause processor(s) 1204 to identify and validate change points in a plurality of concentration data points obtained from an assay.

System 1200 may be configured to receive the plurality of concentration data points from a concentration data storage 1206 (e.g., from one or more computers used by laboratory analysts to store the plurality of concentration data points obtained from the assay). In addition, system 1200 may be configured to receive assay parameter data from an assay parameter storage 1208. The assay parameter data may comprise information about assay parameters (e.g., environmental factors, equipment factors, and/or human factors) associated with the plurality of concentration data points obtained from the assay.

In some embodiments, system 1200 may be coupled to a user interface 1210. Optionally, user interface 1210 may be a component of system 1200. When processor(s) 1204 identify and validate significant change points in a plurality of concentration data points, processor(s) 1204 may cause user interface 1210 to output information about the significant change points. For example, processor(s) 1204 may be configured to cause user interface 1210 to display one or more plots of the plurality of concentration data points and to indicate the location(s) of significant change point(s) on said plots. If assay parameter data corresponding to significant change point(s) is available, processor(s) 1204 may be configured to cause user interface 1210 to provide the corresponding assay parameter data to users, so that users may use the assay parameter data to evaluate root causes of the changes in the plurality of concentration data points.

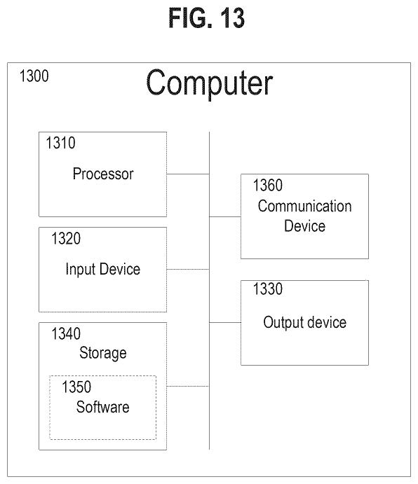

In some embodiments, a system for identifying and validating change points in a plurality of concentration data points may be or may comprise a computer system. FIG. 13 illustrates an example of a computing system. Computer 1300 may be involved in executing one or more of the methods described herein. Computer 1300 can be a host computer connected to a network. Computer 1300 can be a client computer or a server. As shown in FIG. 13, computer 1300 can be any suitable type of microprocessor-based device, such as a personal computer, workstation, server, or handheld computing device, such as a phone or tablet. The computer can include, for example, one or more of processor 1310, input device 1320, output device 1330, storage 1340, and communication device 1360. Input device 1320 and output device 1330 can correspond to those described above and can either be connectable or integrated with the computer.

Input device 1320 can be any suitable device that provides input, such as a touch screen or monitor, keyboard, mouse, or voice-recognition device. Output device 1330 can be any suitable device that provides an output, such as a touch screen, monitor, printer, disk drive, or speaker.

Storage 1340 can be any suitable device that provides storage, such as an electrical, magnetic, or optical memory, including a random-access memory (RAM), cache, hard drive, CD-ROM drive, tape drive, or removable storage disk. Communication device 1360 can include any suitable device capable of transmitting and receiving signals over a network, such as a network interface chip or card. The components of the computer can be connected in any suitable manner, such as via a physical bus or wirelessly. Storage 1340 can be a non-transitory computer-readable storage medium comprising one or more programs, which, when executed by one or more processors, such as processor 1310, cause the one or more processors to execute methods described herein.

Software 1350, which can be stored in storage 1340 and executed by processor 1310, can include, for example, the programming that embodies the functionality of the present disclosure (e.g., as embodied in the systems, computers, servers, and/or devices as described above). In one or more examples, software 1350 can include a combination of servers such as application servers and database servers.

Software 1350 can also be stored and/or transported within any computer-readable storage medium for use by or in connection with an instruction execution system, apparatus, or device, such as those described above, that can fetch and execute instructions associated with the software from the instruction execution system, apparatus, or device. In the context of this disclosure, a computer-readable storage medium can be any medium, such as storage 1340, that can contain or store programming for use by or in connection with an instruction execution system, apparatus, or device.

Software 1350 can also be propagated within any transport medium for use by or in connection with an instruction execution system, apparatus, or device, such as those described above, that can fetch and execute instructions associated with the software from the instruction execution system, apparatus, or device. In the context of this disclosure, a transport medium can be any medium that can communicate, propagate, or transport programming for use by or in connection with an instruction execution system, apparatus, or device. The transport-readable medium can include but is not limited to, an electronic, magnetic, optical, electromagnetic, or infrared wired or wireless propagation medium.

Computer 1300 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Computer 1300 can implement any operating system suitable for operating on the network. Software 1350 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

### Examples of Significant Change Points

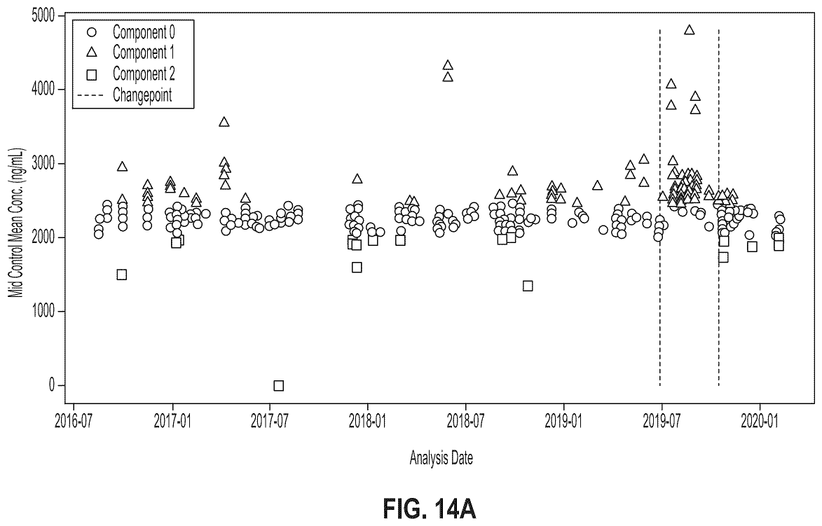

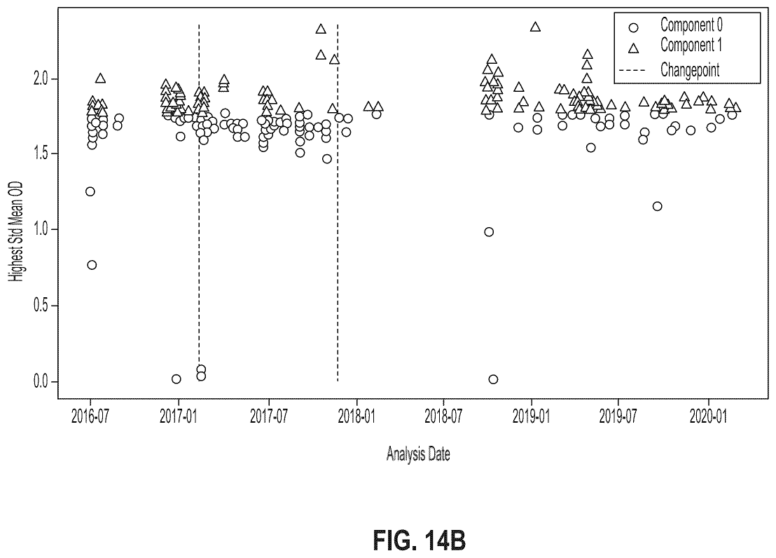

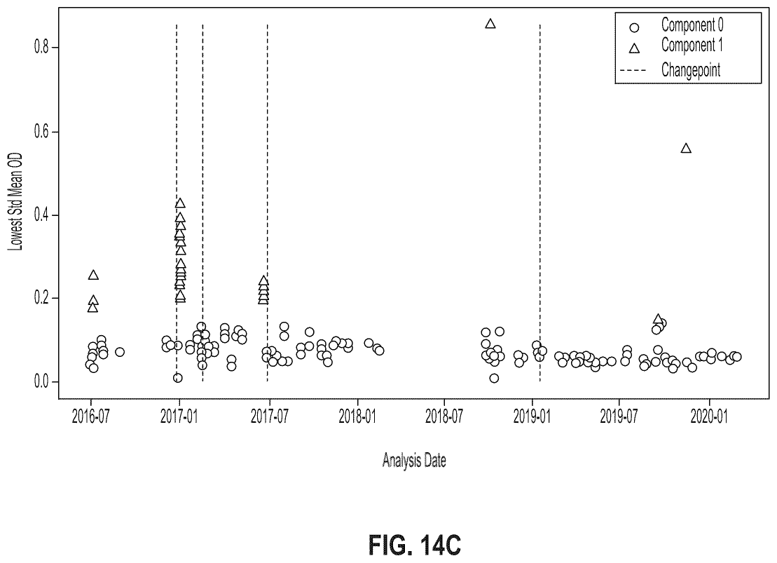

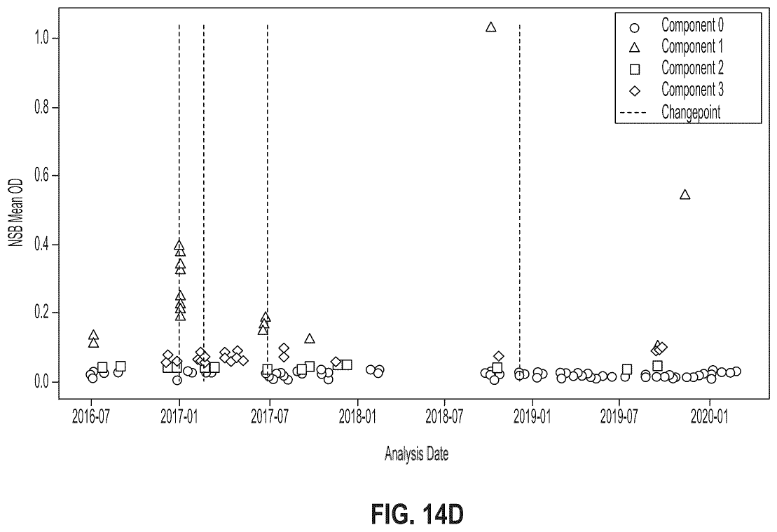

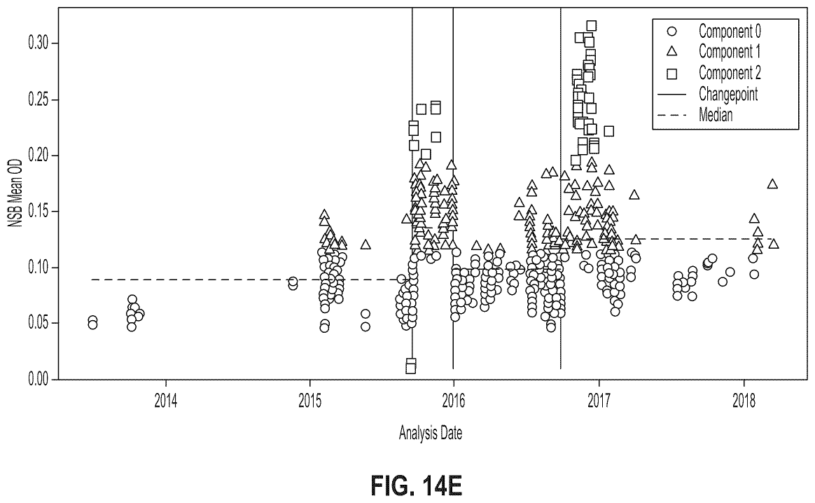

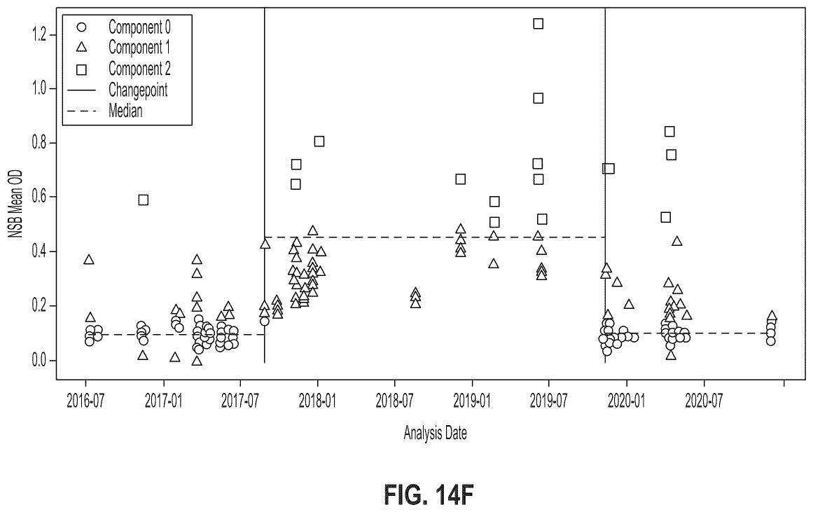

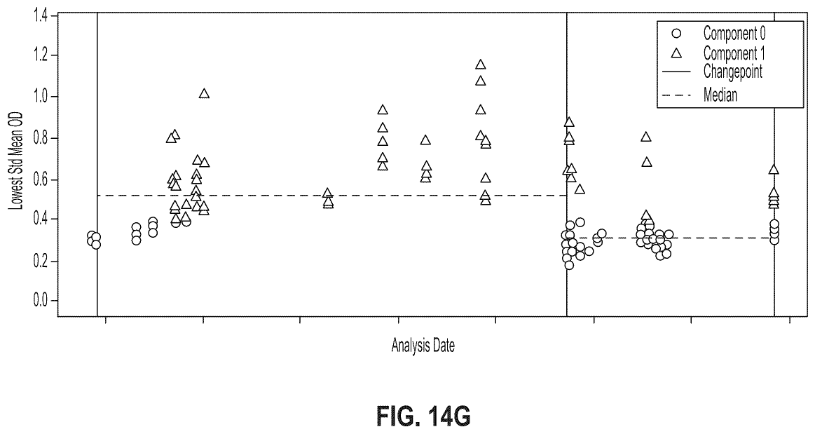

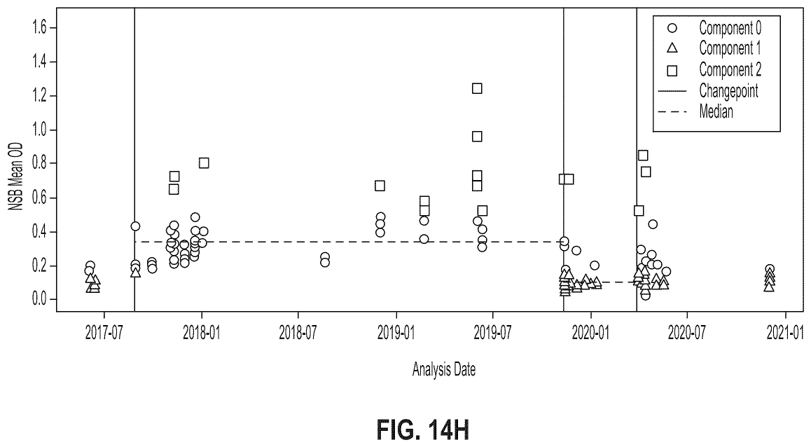

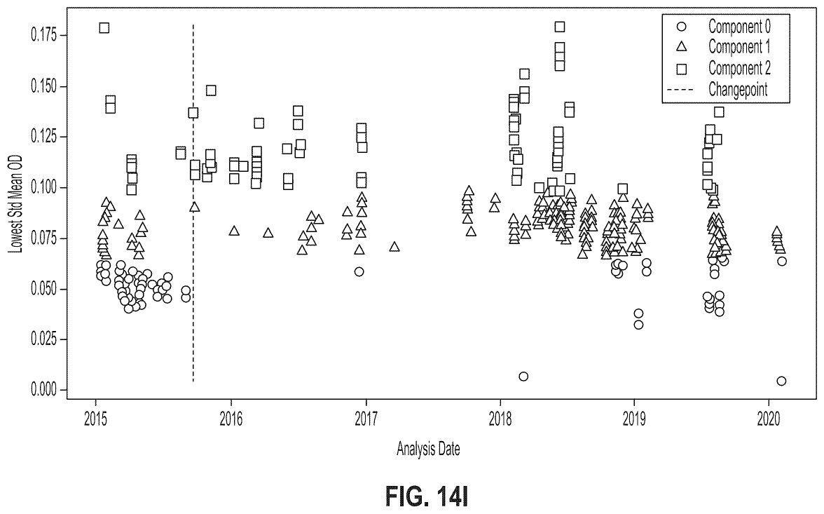

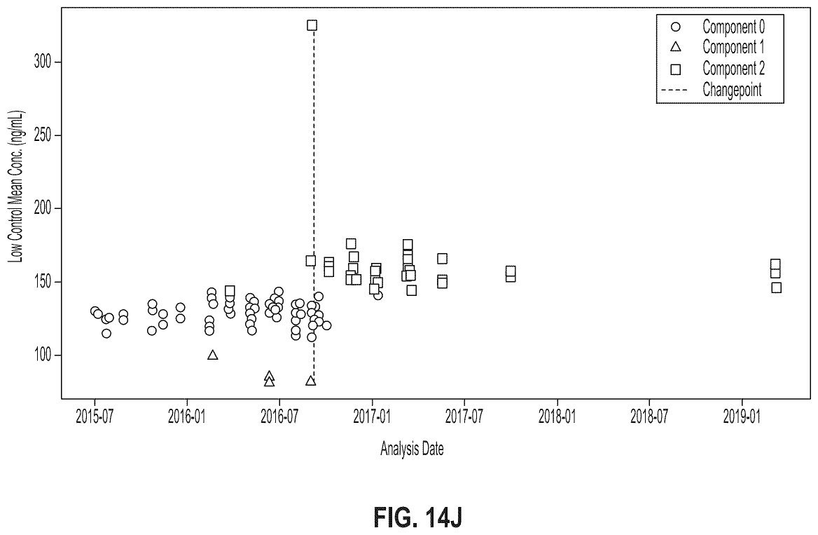

Various examples of significant change points in a plurality of concentration data points that have been identified using the provided methods are provided in FIGS. 14A-14J. As shown, significant change points in a plurality of concentration data points may be locations in the plurality of concentration data points where statistical characteristics of the distribution concentration data points notably change or shift. The disclosed methods allow such change points to be efficiently and accurately located in concentration data obtained from an assay, even when said data is noisy and irregular.

### Example Embodiments

Embodiments disclosed herein may include:
1. A method for determining a cause of a significant statistical change in a plurality of concentration data points obtained from an assay, the method comprising:
   performing a plurality of instances of the assay to obtain the plurality of concentration data points;
   receiving assay information comprising the plurality of concentration data points and a plurality of assay parameters, wherein each assay parameter of the plurality of assay parameters is associated with an instance of the plurality of instances of performing the assay;
   identifying a significant change point that corresponds to a location in the plurality of concentration data points at which one or more statistical characteristics of the plurality of concentration data points change by more than a threshold value;
   correlating one or more assay parameters of the plurality of assay parameters with the identified significant change point by identifying an instance of the plurality of instances of performing the assay that corresponds to the location of the significant change point in the plurality of concentration data points; and
   determining the cause for the change in the one or more statistical characteristics of the plurality of concentration data points at the identified significant change point based on the correlation between the one or more assay parameters and the significant change point.
2. The method of embodiment 1, wherein the assay is configured to measure a concentration of an analyte in a sample.
3. The method of embodiment 2, wherein the analyte is a therapeutic analyte.
4. The method of embodiment 2 or 3, wherein the analyte is a therapeutic polypeptide.
5. The method of any one of embodiments 2-4, wherein the analyte is an antibody or a fragment thereof.
6. The method of any one of embodiments 2-5, wherein the sample is a cell culture sample or a derivative thereof.
7. The method of any one of embodiments 1-7, wherein the assay is an immunoassay.
8. The method of any one of embodiments 1-7, wherein the assay is a competitive assay.
9. The method of any one of embodiments 1-7, wherein the assay is a non-competitive assay.
10. The method of any one of embodiments 1-7, wherein the assay is a non-homogenous assay.
11. The method of any one of embodiments 1-7, wherein the assay is a homogenous assay.
12. The method of any one of embodiments 1-10, wherein the assay is an ELISA assay.
13. The method of embodiment 12, wherein the ELISA assay is a direct ELISA assay.
14. The method of embodiment 12, wherein the ELISA assay is a sandwich ELISA assay.
15. The method of embodiment 12, wherein the ELISA assay is a competitive ELISA assay.
16. The method of any one of embodiments 1-15, wherein performing the plurality of instances of the assay comprises performing two or more of the plurality of instances at two or more times.
17. The method of embodiment 16, wherein the two or more times constitute a time course of at least about one week.
18. The method of any one of embodiments 1-17, wherein performing the plurality of instances of the assay comprises performing two or more of the plurality of instances simultaneously.
19. The method of any one of embodiments 1-18, wherein the plurality of concentration data points comprises data points pertaining to a concentration a target analyte.
20. The method of any one of embodiments 1-19, wherein the plurality of concentration data points comprises data points pertaining to a concentration of a control.
21. The method of embodiment 20, wherein the control is a negative control, non-specific binding control, blank control, detection antibody control, negative matrix control, or positive control.
22. The method of any one of embodiments 1-21, wherein the plurality of concentration data points comprises data points pertaining to a solution concentration.
23. The method of any one of embodiments 1-22, wherein the plurality of concentration data points comprises data points pertaining to an absolute amount of a target analyte.
24. The method of any one of embodiments 1-23, wherein the plurality of concentration data points comprises data points pertaining to a measurement associated with concentration.
25. The method of embodiment 24, wherein the measurement associated with concentration is an optical density (OD) measurement.
26. The method of any one of embodiments 1-25, wherein the plurality of concentration data points comprises data points pertaining to a mean, lowest standard deviation mean, highest standard deviation mean, or middle control concentration.
27. The method of any one of embodiments 1-26, wherein the significant change point reflects inter-assay variability.
28. The method of any one of embodiments 1-27, wherein the significant change point reflects intra-assay variability.
29. The method of any one of embodiments 1-28, wherein two or more concentration data points of the plurality of concentration data points are in the same format.
30. The method of any one of embodiments 1-29, wherein the one or more assay parameters correlated with the significant change point comprise an environmental factor.
31. The method of embodiment 30, wherein the environmental factor comprises a temperature, humidity, light, or contaminant.
32. The method of any one of embodiments 1-31, wherein the one or more assay parameters correlated with significant change points comprise an equipment factor.
33. The method of embodiment 32, wherein the equipment factor comprises a reagent change, reagent aging, reagent expiration, reagent contamination, reagent failure, hardware change, hardware aging, hardware contamination, hardware failure, instrument change, instrument failure, or instrument calibration.
34. The method of any one of embodiments 1-33, wherein the one or more assay parameters correlated with the significant change point comprise a human factor associated with one or more humans who performed or assisted in performing the assay.
35. The method of embodiment 34, wherein the human factor comprises performance variability, performance error, or operator change.
36. The method of any one of embodiments 1-35, wherein identifying the significant change point comprises determining an expected change point population in the plurality of concentration data points.
37. The method of any one of embodiments 1-36, wherein identifying the significant change point comprises:
   selecting a first segment of concentration data points of the plurality of concentration data points;
   determining a first median value associated with the first segment of concentration data points;
   selecting a second segment of concentration data points of the plurality of concentration data points, wherein the concentration data points in the second segment are consecutive to the concentration data points in the first segment;
   determining a second median value associated with the second segment of concentration data points;
   comparing the first median value to the second median value; and
   determining, based on the comparison between the first median value and the second median value, whether a candidate change point is located between the first segment and the second segment.
38. The method of embodiment 37, wherein the first segment and the second segment comprise at least a threshold number of concentration data points.
39. The method of embodiment 38, comprising receiving the threshold number of concentration data points from a user.
40. The method of embodiment 38, wherein the threshold number of concentration data points is determined based on the assay.
41. The method of any one of embodiments 37-40, comprising, for the first segment and the second segment:
   generating one or more mean values;
   generating one or more data point clusters, wherein each data point cluster is associated with a mean value of the one or more mean values and comprises concentration data points in the segment that are closest to the associated mean value;
   updating the mean value for each data point cluster of the one or more data point clusters, wherein updating the mean value for a data point cluster comprises identifying a centroid of the data point cluster; and
   iteratively repeating the steps of generating one or more data point clusters and updating the mean values for each data point cluster until the mean values for each data point cluster no longer change.
42. The method of any one of embodiments 37-40, comprising generating one or more data point clusters within each of the first segment and the second segment, wherein the concentration data points in each data point cluster are normally distributed and have a unique mean value and a unique standard deviation value.
43. The method of embodiment 41 or 42, comprising identifying, for the first segment and the second segment, a principal data point cluster of the one or more data point clusters, wherein the principal data point cluster comprises at least a threshold percentage of a total number of concentration data points in the segment.
44. The method of embodiment 43, comprising determining, for the candidate change point, a divergence value, wherein the divergence value measures a statistical difference between the principal data point cluster in the first segment and the principal data point cluster in the second segment.
45. The method of embodiment 44, wherein the divergence value is a Jensen-Shannon divergence.
46. The method of any one of embodiments 43-45, comprising determining, for the candidate change point, a median change value, wherein the median change value measures a difference between the first median value associated with the first segment and the second median value associated with the second segment.
47. The method of embodiment 46, comprising determining whether the one or more statistical characteristics of the plurality of concentration data points change by more than a threshold value by determining a weighted combination of the divergence value and the median change value.
48. The method of embodiment 47, wherein the weighted combination of the divergence value and the median change value is characterized by a weight parameter.
49. The method of embodiment 48, comprising receiving the weight parameter from a user.
50. The method of embodiment 48 or 49, wherein the weight parameter is determined based on the assay.
51. The method of any one of embodiments 1-50, comprising performing a second plurality of instances of the assay after determining the cause for the change in the one or more statistical characteristics of the plurality of concentration data points at the identified significant change point.
52. The method of embodiment 50, wherein the second plurality of instances of the assay are performed with assay parameters that match the one or more assay parameters that are correlated with the identified significant change point.
53. The method of embodiment 50, wherein the second plurality of instances of the assay are performed with assay parameters that match assay parameters associated with instances of performing the assay that occurred prior to the identified significant change point.
54. The method of any one of embodiments 1-53, comprising deleting one or more concentration data points of the plurality of concentration data points that correspond to instances of the plurality of instances of performing the assay that occurred after the identified significant change point.
55. A system for determining a cause of a significant statistical change in a plurality of concentration data points obtained from an assay, the system comprising one or more processors configured to:
   receive assay information comprising the plurality of concentration data points obtained by performing a plurality of instances of the assay and a plurality of assay parameters, wherein each assay parameter of the plurality of assay parameters is associated with an instance of the plurality of instances of performing the assay;
   identify a significant change point that corresponds to a location in the plurality of concentration data points at which one or more statistical characteristics of the plurality of concentration data points change by more than a threshold value;
   correlate one or more assay parameters of the plurality of assay parameters with the identified significant change point by identifying an instance of the plurality of instances of performing the assay that corresponds to the location of the significant change point in the plurality of concentration data points; and
   determine the cause for the change in the one or more statistical characteristics of the plurality of concentration data points at the identified significant change point based on the correlation between the one or more assay parameters and the significant change point.
56. The system of embodiment 55, wherein the assay is configured to measure a concentration of an analyte in a sample.
57. The system of embodiment 56, wherein the analyte is a therapeutic analyte.
58. The system of embodiment 56 or 57, wherein the analyte is a therapeutic polypeptide.
59. The system of any one of embodiments 56-58, wherein the analyte is an antibody or a fragment thereof.
60. The system of any one of embodiments 56-59, wherein the sample is a cell culture sample or a derivative thereof.
61. The system of any one of embodiments 55-60, wherein the assay is an immunoassay.
62. The system of any one of embodiments 55-60, wherein the assay is a competitive assay.
63. The system of any one of embodiments 55-60, wherein the assay is a non-competitive assay.
64. The system of any one of embodiments 55-60, wherein the assay is a non-homogenous assay.
65. The system of any one of embodiments 55-60, wherein the assay is a homogenous assay.
66. The system of any one of embodiments 55-65, wherein the assay is an ELISA assay.
67. The system of embodiment 66, wherein the ELISA assay is a direct ELISA assay.
68. The system of embodiment 66, wherein the ELISA assay is a sandwich ELISA assay.
69. The system of embodiment 66, wherein the ELISA assay is a competitive ELISA
70. The system of any one of embodiments 55-69, wherein performing the plurality of instances of the assay comprises performing two or more of the plurality of instances at two or more times.
71. The system of embodiment 70, wherein the two or more times constitute a time course of at least about one week.
72. The system of any one of embodiments 55-71, wherein performing the plurality of instances of the assay comprises performing two or more of the plurality of instances simultaneously.
73. The system of any one of embodiments 55-72, wherein the plurality of concentration data points comprises data points pertaining to a concentration a target analyte.
74. The system of any one of embodiments 55-73, wherein the plurality of concentration data points comprises data points pertaining to a concentration of a control.
75. The system of embodiment 74, wherein the control is a negative control, non-specific binding control, blank control, detection antibody control, negative matrix control, or positive control.
76. The system of any one of embodiments 55-75, wherein the plurality of concentration data points comprises data points pertaining to a solution concentration.
77. The system of any one of embodiments 55-76, wherein the plurality of concentration data points comprises data points pertaining to an absolute amount of a target analyte.
78. The system of any one of embodiments 55-77, wherein the plurality of concentration data points comprises data points pertaining to a measurement associated with concentration.
79. The system of embodiment 78, wherein the measurement associated with concentration is an optical density (OD) measurement.
80. The system of any one of embodiments 55-79, wherein the plurality of concentration data points comprises data points pertaining to a mean, lowest standard deviation mean, highest standard deviation mean, or middle control concentration.
81. The system of any one of embodiments 55-80, wherein the significant change point reflects inter-assay variability.
82. The system of any one of embodiments 55-81, wherein the significant change point reflects intra-assay variability.
83. The system of any one of embodiments 55-82, wherein two or more concentration data points of the plurality of concentration data points are in the same format.
84. The system of any one of embodiments 55-83, wherein the one or more assay parameters correlated with the significant change point comprise an environmental factor.
85. The system of embodiment 84, wherein the environmental factor comprises a temperature, humidity, light, or contaminant.
86. The system of any one of embodiments 55-85, wherein the one or more assay parameters correlated with significant change points comprise an equipment factor.
87. The system of embodiment 86, wherein the equipment factor comprises a reagent change, reagent aging, reagent expiration, reagent contamination, reagent failure, hardware change, hardware aging, hardware contamination, hardware failure, instrument change, instrument failure, or instrument calibration.
88. The system of any one of embodiments 55-87, wherein the one or more assay parameters correlated with the significant change point comprise a human factor associated with one or more humans who performed or assisted in performing the assay.
89. The system of embodiment 88, wherein the human factor comprises performance variability, performance error, or operator change.
90. The system of any one of embodiments 55-89, wherein identifying the significant change point comprises determining an expected change point population in the plurality of concentration data points.
91. The system of any one of embodiments 55-90, wherein identifying the significant change point comprises:
   selecting a first segment of concentration data points of the plurality of concentration data points;
   determining a first median value associated with the first segment of concentration data points;
   selecting a second segment of concentration data points of the plurality of concentration data points, wherein the concentration data points in the second segment are consecutive to the concentration data points in the first segment;
   determining a second median value associated with the second segment of concentration data points;
   comparing the first median value to the second median value; and
   determining, based on the comparison between the first median value and the second median value, whether a candidate change point is located between the first segment and the second segment.
92. The system of embodiment 91, wherein the first segment and the second segment comprise at least a threshold number of concentration data points.
93. The system of embodiment 92, wherein the one or more processors are configured to receive the threshold number of concentration data points from a user.
94. The system of embodiment 93, wherein the threshold number of concentration data points is determined based on the assay.
95. The system of any one of embodiments 91-94, wherein the one or more processors are configured to, for the first segment and the second segment:
   generate one or more mean values;
   generate one or more data point clusters, wherein each data point cluster is associated with a mean value of the one or more mean values and comprises concentration data points in the segment that are closest to the associated mean value;
   update the mean value for each data point cluster of the one or more data point clusters, wherein updating the mean value for a data point cluster comprises identifying a centroid of the data point cluster; and
   iteratively repeat the steps of generating one or more data point clusters and updating the mean values for each data point cluster until the mean values for each data point cluster no longer change.
96. The system of any one of embodiments 91-94, wherein the one or more processors are configured to generate one or more data point clusters within each of the first segment and the second segment, wherein the concentration data points in each data point cluster are normally distributed and have a unique mean value and a unique standard deviation value.
97. The system of embodiment 95 or 96, wherein the one or more processors are configured to identify, for the first segment and the second segment, a principal data point cluster of the one or more data point clusters, wherein the principal data point cluster comprises at least a threshold percentage of a total number of concentration data points in the segment.
98. The system of embodiment 97, wherein the one or more processors are configured to determine, for the candidate change point, a divergence value, wherein the divergence value measures a statistical difference between the principal data point cluster in the first segment and the principal data point cluster in the second segment.
99. The system of embodiment 98, wherein the divergence value is a Jensen-Shannon divergence.
100. The system of any one of embodiments 97-99, wherein the one or more processors are configured to determine, for the candidate change point, a median change value, wherein the median change value measures a difference between the first median value associated with the first segment and the second median value associated with the second segment.
101. The system of embodiment 100, wherein the one or more processors are configured to determine whether the one or more statistical characteristics of the plurality of concentration data points change by more than a threshold value by determining a weighted combination of the divergence value and the median change value.
102. The system of embodiment 101, wherein the weighted combination of the divergence value and the median change value is characterized by a weight parameter.
103. The system of embodiment 102, wherein the one or more processors are configured to receive the weight parameter from a user.
104. The system of embodiment 102 or 103, wherein the weight parameter is determined based on the assay.
105. The system of any one of embodiments 55-104, wherein the one or more processors are configured to delete one or more concentration data points of the plurality of concentration data points that correspond to instances of the plurality of instances of performing the assay that occurred after the identified significant change point.
106. A non-transitory computer readable storage medium storing instructions for determining a cause of a significant statistical change in a plurality of concentration data points obtained from an assay, wherein the instructions are configured to be executed by one or more processors of an electronic device to cause the device to:
   receive assay information comprising the plurality of concentration data points obtained by performing a plurality of instances of the assay and a plurality of assay parameters, wherein each assay parameter of the plurality of assay parameters is associated with an instance of the plurality of instances of performing the assay;
   identify a significant change point that corresponds to a location in the plurality of concentration data points at which one or more statistical characteristics of the plurality of concentration data points change by more than a threshold value;
   correlate one or more assay parameters of the plurality of assay parameters with the identified significant change point by identifying an instance of the plurality of instances of performing the assay that corresponds to the location of the significant change point in the plurality of concentration data points; and
   determine the cause for the change in the one or more statistical characteristics of the plurality of concentration data points at the identified significant change point based on the correlation between the one or more assay parameters and the significant change point.
107. The non-transitory computer readable storage medium of embodiment 106, wherein the assay is configured to measure a concentration of an analyte in a sample.
108. The non-transitory computer readable storage medium of embodiment 107, wherein the analyte is a therapeutic analyte.
109. The non-transitory computer readable storage medium of embodiment 107 or 108, wherein the analyte is a therapeutic polypeptide.
110. The non-transitory computer readable storage medium of any one of embodiments 107-109, wherein the analyte is an antibody or a fragment thereof.
111. The non-transitory computer readable storage medium of any one of embodiments 107-110, wherein the sample is a cell culture sample or a derivative thereof.
112. The non-transitory computer readable storage medium of any one of embodiments 106-111, wherein the assay is an immunoassay.
113. The non-transitory computer readable storage medium of any one of embodiments 106-111, wherein the assay is a competitive assay.
114. The non-transitory computer readable storage medium of any one of embodiments 106-111, wherein the assay is a non-competitive assay.
115. The non-transitory computer readable storage medium of any one of embodiments 106-111, wherein the assay is a non-homogenous assay.
116. The non-transitory computer readable storage medium of any one of embodiments 106-111, wherein the assay is a homogenous assay.
117. The non-transitory computer readable storage medium of any one of embodiments 106-116, wherein the assay is an ELISA assay.
118. The non-transitory computer readable storage medium of embodiment 117, wherein the ELISA assay is a direct ELISA assay.
119. The non-transitory computer readable storage medium of embodiment 117, wherein the ELISA assay is a sandwich ELISA assay.
120. The non-transitory computer readable storage medium of embodiment 117, wherein the ELISA assay is a competitive ELISA assay.
121. The non-transitory computer readable storage medium of any one of embodiments 106-120, wherein performing the plurality of instances of the assay comprises performing two or more of the plurality of instances at two or more times.
122. The non-transitory computer readable storage medium of embodiment 121, wherein the two or more times constitute a time course of at least about one week.
123. The non-transitory computer readable storage medium of any one of embodiments 106-122, wherein performing the plurality of instances of the assay comprises performing two or more of the plurality of instances simultaneously.
124. The non-transitory computer readable storage medium of any one of embodiments 106-123, wherein the plurality of concentration data points comprises data points pertaining to a concentration a target analyte.
125. The non-transitory computer readable storage medium of any one of embodiments 106-124, wherein the plurality of concentration data points comprises data points pertaining to a concentration of a control.
126. The non-transitory computer readable storage medium of embodiment 125, wherein the control is a negative control, non-specific binding control, blank control, detection antibody control, negative matrix control, or positive control.
127. The non-transitory computer readable storage medium of any one of embodiments 106-126, wherein the plurality of concentration data points comprises data points pertaining to a solution concentration.
128. The non-transitory computer readable storage medium of any one of embodiments 106-127, wherein the plurality of concentration data points comprises data points pertaining to an absolute amount of a target analyte.
129. The non-transitory computer readable storage medium of any one of embodiments 106-128, wherein the plurality of concentration data points comprises data points pertaining to a measurement associated with concentration.
130. The non-transitory computer readable storage medium of embodiment 129, wherein the measurement associated with concentration is an optical density (OD) measurement.
131. The non-transitory computer readable storage medium of any one of embodiments 106-130, wherein the plurality of concentration data points comprises data points pertaining to a mean, lowest standard deviation mean, highest standard deviation mean, or middle control concentration.
132. The non-transitory computer readable storage medium of any one of embodiments 106-131, wherein the significant change point reflects inter-assay variability.
133. The non-transitory computer readable storage medium of any one of embodiments 106-132, wherein the significant change point reflects intra-assay variability.
134. The non-transitory computer readable storage medium of any one of embodiments 106-133, wherein two or more concentration data points of the plurality of concentration data points are in the same format.
135. The non-transitory computer readable storage medium of any one of embodiments 106-134, wherein the one or more assay parameters correlated with the significant change point comprise an environmental factor.
136. The non-transitory computer readable storage medium of embodiment 135, wherein the environmental factor comprises a temperature, humidity, light, or contaminant.
137. The non-transitory computer readable storage medium of any one of embodiments 106-136, wherein the one or more assay parameters correlated with significant change points comprise an equipment factor.
138. The non-transitory computer readable storage medium of embodiment 137, wherein the equipment factor comprises a reagent change, reagent aging, reagent expiration, reagent contamination, reagent failure, hardware change, hardware aging, hardware contamination, hardware failure, instrument change, instrument failure, or instrument calibration.
139. The non-transitory computer readable storage medium of any one of embodiments 106-138, wherein the one or more assay parameters correlated with the significant change point comprise a human factor associated with one or more humans who performed or assisted in performing the assay.
140. The non-transitory computer readable storage medium of embodiment 139, wherein the human factor comprises performance variability, performance error, or operator change.
141. The non-transitory computer readable storage medium of any one of embodiments 106-140, wherein identifying the significant change point comprises determining an expected change point population in the plurality of concentration data points.
142. The non-transitory computer readable storage medium of any one of embodiments 106-141, wherein identifying the significant change point comprises:
   selecting a first segment of concentration data points of the plurality of concentration data points;
   determining a first median value associated with the first segment of concentration data points;
   selecting a second segment of concentration data points of the plurality of concentration data points, wherein the concentration data points in the second segment are consecutive to the concentration data points in the first segment;
   determining a second median value associated with the second segment of concentration data points;
   comparing the first median value to the second median value; and
   determining, based on the comparison between the first median value and the second median value, whether a candidate change point is located between the first segment and the second segment.
143. The non-transitory computer readable storage medium of embodiment 142, wherein the first segment and the second segment comprise at least a threshold number of concentration data points.
144. The non-transitory computer readable storage medium of embodiment 143, wherein the instructions, when executed by the one or more processors of the electronic device, are configured to cause the electronic device to receive the threshold number of concentration data points from a user.
145. The non-transitory computer readable storage medium of embodiment 144, wherein the threshold number of concentration data points is determined based on the assay.
146. The non-transitory computer readable storage medium of any one of embodiments 142-145, wherein the instructions, when executed by the one or more processors of the electronic device, are configured to cause the device to, for the first segment and the second segment:
   generate one or more mean values;
   generate one or more data point clusters, wherein each data point cluster is associated with a mean value of the one or more mean values and comprises concentration data points in the segment that are closest to the associated mean value;
   update the mean value for each data point cluster of the one or more data point clusters, wherein updating the mean value for a data point cluster comprises identifying a centroid of the data point cluster; and
   iteratively repeat the steps of generating one or more data point clusters and updating the mean values for each data point cluster until the mean values for each data point cluster no longer change.
147. The non-transitory computer readable storage medium of any one of embodiments 142-145, wherein the instructions, when executed by the one or more processors of the electronic device, are configured to cause the device to generate one or more data point clusters within each of the first segment and the second segment, wherein the concentration data points in each data point cluster are normally distributed and have a unique mean value and a unique standard deviation value.
148. The non-transitory computer readable storage medium of embodiment 146 or 147, wherein the instructions, when executed by the one or more processors of the electronic device, are configured to cause the device to identify, for the first segment and the second segment, a principal data point cluster of the one or more data point clusters, wherein the principal data point cluster comprises at least a threshold percentage of a total number of concentration data points in the segment.
149. The non-transitory computer readable storage medium of embodiment 148, wherein the instructions, when executed by the one or more processors of the electronic device, are configured to cause the device to determine, for the candidate change point, a divergence value, wherein the divergence value measures a statistical difference between the principal data point cluster in the first segment and the principal data point cluster in the second segment.
150. The non-transitory computer readable storage medium of embodiment 149, wherein the divergence value is a Jensen-Shannon divergence.
151. The non-transitory computer readable storage medium of any one of embodiments 148-150, wherein the instructions, when executed by the one or more processors of the electronic device, are configured to cause the device to determine, for the candidate change point, a median change value, wherein the median change value measures a difference between the first median value associated with the first segment and the second median value associated with the second segment.
152. The non-transitory computer readable storage medium of embodiment 151, wherein the instructions, when executed by the one or more processors of the electronic device, are configured to cause the device to determine whether the one or more statistical characteristics of the plurality of concentration data points change by more than a threshold value by determining a weighted combination of the divergence value and the median change value.
153. The non-transitory computer readable storage medium of embodiment 152, wherein the weighted combination of the divergence value and the median change value is characterized by a weight parameter.
154. The non-transitory computer readable storage medium of embodiment 153, wherein the instructions, when executed by the one or more processors of the electronic device, are configured to cause the device to receive the weight parameter from a user.
155. The non-transitory computer readable storage medium of embodiment 153 or 154, wherein the weight parameter is determined based on the assay.
156. The non-transitory computer readable storage medium of any one of embodiments 106-155, wherein the instructions, when executed by the one or more processors of the electronic device, are configured to cause the device to delete one or more concentration data points of the plurality of concentration data points that correspond to instances of the plurality of instances of performing the assay that occurred after the identified significant change point.

The description provides preferred example embodiments only, and is not intended to limit the scope, applicability or configuration of the disclosure. Rather, the description of the preferred example embodiments will provide those skilled in the art with an enabling description for implementing various embodiments. It is understood that various changes may be made in the function and arrangement of elements without departing from the spirit and scope as set forth in the appended claims.

Specific details are given in the description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

### CONCLUSION

The foregoing description, for the purpose of explanation, has been described with reference to specific embodiments and/or examples. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

Any of the systems, methods, techniques, and/or features disclosed herein may be combined, in whole or in part, with any other systems, methods, techniques, and/or features disclosed herein.

1. A system for determining a cause of a significant statistical change in a plurality of concentration data points obtained from an assay, the system comprising one or more processors configured to:
receive assay information comprising the plurality of concentration data points obtained by performing a plurality of instances of the assay and a plurality of assay parameters, wherein each assay parameter of the plurality of assay parameters is associated with an instance of the plurality of instances of performing the assay;
identify a significant change point that corresponds to a location in the plurality of concentration data points at which one or more statistical characteristics of the plurality of concentration data points change by more than a threshold value;
correlate one or more assay parameters of the plurality of assay parameters with the identified significant change point by identifying an instance of the plurality of instances of performing the assay that corresponds to the location of the significant change point in the plurality of concentration data points; and
determine the cause for the change in the one or more statistical characteristics of the plurality of concentration data points at the identified significant change point based on the correlation between the one or more assay parameters and the significant change point.

2. The system of claim 1, wherein the assay is configured to measure a concentration of an analyte in a sample.

3. The system of claim 1, wherein the one or more assay parameters correlated with the significant change point comprise an environmental factor comprising a temperature, humidity, light, or contaminant.

4. The system of claim 1, wherein the one or more assay parameters correlated with significant change points comprise an equipment factor.

5. The system of claim 4, wherein the equipment factor comprises a reagent change, reagent aging, reagent expiration, reagent contamination, reagent failure, hardware change, hardware aging, hardware contamination, hardware failure, instrument change, instrument failure, or instrument calibration.

6. The system of claim 1, wherein the one or more assay parameters correlated with the significant change point comprise a human factor associated with one or more humans who performed or assisted in performing the assay.

7. The system of claim 6, wherein the human factor comprises performance variability, performance error, or operator change.

8. The system of claim 1, wherein identifying the significant change point comprises determining an expected change point population in the plurality of concentration data points.

9. The system of claim 1, wherein identifying the significant change point comprises:
selecting a first segment of concentration data points of the plurality of concentration data points;
determining a first median value associated with the first segment of concentration data points;
selecting a second segment of concentration data points of the plurality of concentration data points, wherein the concentration data points in the second segment are consecutive to the concentration data points in the first segment;
determining a second median value associated with the second segment of concentration data points;
comparing the first median value to the second median value; and
determining, based on the comparison between the first median value and the second median value, whether a candidate change point is located between the first segment and the second segment.

10. The system of claim 9, wherein the first segment and the second segment comprise at least a threshold number of concentration data points.

11. The system of claim 10, wherein the one or more processors are configured to receive the threshold number of concentration data points from a user.

12. The system of claim 11, wherein the threshold number of concentration data points is determined based on the assay.

13. The system of claim 9, wherein the one or more processors are configured to, for the first segment and the second segment:
generate one or more mean values;
generate one or more data point clusters, wherein each data point cluster is associated with a mean value of the one or more mean values and comprises concentration data points in the segment that are closest to the associated mean value;
update the mean value for each data point cluster of the one or more data point clusters, wherein updating the mean value for a data point cluster comprises identifying a centroid of the data point cluster; and
iteratively repeat the steps of generating one or more data point clusters and updating the mean values for each data point cluster until the mean values for each data point cluster no longer change.

14. The system of claim 9, wherein the one or more processors are configured to generate one or more data point clusters within each of the first segment and the second segment, wherein the concentration data points in each data point cluster are normally distributed and have a unique mean value and a unique standard deviation value.

15. The system of claim 13, wherein the one or more processors are configured to identify, for the first segment and the second segment, a principal data point cluster of the one or more data point clusters, wherein the principal data point cluster comprises at least a threshold percentage of a total number of concentration data points in the segment.

16. The system of claim 15, wherein the one or more processors are configured to determine, for the candidate change point, a divergence value, wherein the divergence value measures a statistical difference between the principal data point cluster in the first segment and the principal data point cluster in the second segment.

17. The system of claim 16, wherein the divergence value is a Jensen-Shannon divergence.

18. The system of claim 15, wherein the one or more processors are configured to determine, for the candidate change point, a median change value, wherein the median change value measures a difference between the first median value associated with the first segment and the second median value associated with the second segment.

19. The system of claim 18, wherein the one or more processors are configured to determine whether the one or more statistical characteristics of the plurality of concentration data points change by more than a threshold value by determining a weighted combination of the divergence value and the median change value.

20. The system of claim 1, wherein the one or more processors are configured to delete one or more concentration data points of the plurality of concentration data points that correspond to instances of the plurality of instances of performing the assay that occurred after the identified significant change point.
